(19) United States
(12) Patent Application Publication
(10) Pub. No.: US 2025/0269519 A1
(43) Pub. Date: Aug. 28, 2025

(54) TRANSMISSION ASSEMBLY FOR DRIVING INSTRUMENT INSERTION, AND RELATED DEVICES, SYSTEMS AND METHODS

(71) Applicant: INTUITIVE SURGICAL OPERATIONS, INC., Sunnyvale, CA (US)

(72) Inventor: William A. Burbank, Sandy Hook, CT (US)

(73) Assignee: INTUITIVE SURGICAL OPERATIONS, INC., Sunnyvale, CA (US)

(21) Appl. No.: 19/064,172

(22) Filed: Feb. 26, 2025

Related U.S. Application Data
(60) Provisional application No. 63/558,935, filed on Feb. 28, 2024.

(51) Int. Cl. B25J 9/10; B25J 19/00

(52) U.S. Cl. CPC B25J 9/109; B25J 9/104; B25J 9/106; B25J 19/007

(57) ABSTRACT

A medical instrument comprises a shaft, a movable component coupled to the shaft, a first actuation element coupled to the instrument shaft, a second actuation element coupled to the movable component, and a transmission assembly movably coupled to the instrument shaft. The transmission assembly comprises: a first drive member comprising a drum which is rotatable to drive translation of the instrument shaft relative to the transmission assembly by actuating the first and second actuation elements which are wound around the drum; a second drive member comprising a first rotatable drive shaft which is rotatable to actuate the second actuation element; and a third drive member comprising a second rotatable drive shaft and a bearing coupled to the second rotatable drive shaft. The second actuation element is routed over the bearing between the rotatable drum and the first rotatable drive shaft.

### CROSS-REFERENCE TO RELATED APPLICATIONS

This application claims priority to U.S. Provisional Application No. 63/558,935, filed Feb. 28, 2024, which is incorporated by reference in its entirety.

### FIELD

Aspects of this disclosure relate generally to instruments for use with computer-assisted teleoperated manipulator systems, and related devices, systems, and methods. More specifically, aspects of the disclosure relate to instrument transmission assembly architectures that provide a drive for instrument insertion.

### Introduction

Some remotely-controlled instruments comprise a shaft, an end effector coupled to the shaft, and a transmission assembly coupled to the shaft. The end effector generally comprises one or more functional elements, such as, for example, a jaw mechanism, a stapler, a cutting implement, a camera, an electrode, a sensor, etc., to perform one or more functions of the instrument, such as cutting, sealing, grasping, imaging, etc. The transmission assembly may comprise one or more drive members configured to receive driving forces, torques, or other inputs and transmit the received driving forces, torques, or other inputs to other portions of the instrument, such as the end effector and/or other structures along portions of the shaft (e.g., joints and or wrist mechanisms), to drive degrees of freedom of motion and/or functions of the instrument. The drive members may transmit received driving forces or torques to other portions of the instrument via actuation elements running from the transmission assembly along the instrument shaft. Such actuation elements are generally in the form of tension members such as cables, wires, filaments or the like that are flexible in all directions and generally transmit force by pulling on the actuation element to place it in tension (referred to herein as pullable actuation elements), in the form of more rigid compression members such as rods, sheet metal strips, push-coils, or the like that can transmit force by pushing (or in some cases by both pushing and pulling) on the actuation element (referred to herein as pushable actuation elements), or in the form of members that transmit force by rotation, such as lead screws, drive shafts, or the like (referred to herein as rotatable actuation elements). In some cases, the drive members may convert received driving forces or torques from one form to another, such as, for example, converting received torques into linear forces.

The transmission assembly of such instruments can be configured to be coupled to computer-assisted teleoperated manipulator systems to receive the drive inputs, such as via servo-motor and drive disk interfaces, and/or may be configured to receive input manually, such as via a user operating a wheel, button, trigger, or other mechanism at the force transmission assembly to provide the desired input.

Some remotely-controlled instruments have a degree of freedom of motion comprising linear translation of the instrument shaft relative to a workspace (e.g., a patient's body) along a longitudinal axis of the shaft. Such translation of the instrument shaft, and hence the end effector coupled thereto, may allow for insertion of the end effector into (or advancement further into) the workspace and withdrawal of the end effector from the workspace. This degree of freedom of motion may be referred to herein as an insertion degree of freedom, and the direction along which the translation occurs may be referred to herein as an insertion axis.

Some instruments, and manipulator systems, provide for the insertion degree of freedom of motion by translating the entire instrument, including the transmission assembly, along the insertion axis. For example, for a teleoperated manipulator system, an instrument holder to which the instrument is coupled, may be translatable along a link of a manipulator of the manipulator system. Likewise, for a manually-operated instrument, insertion may occur via movement of the entire instrument including the transmission assembly, via a user generally holding the instrument at least in part by the transmission assembly housing.

However, other instruments and manipulator systems provide for the insertion degree of freedom of the instrument shaft and end effector by providing for relative translation between the transmission assembly of the instrument and the shaft of the instrument, with such relative translation being driven via drive input from the transmission assembly itself. In such systems, the relative translation between the instrument shaft and the transmission assembly causes translation of the instrument shaft relative to the workspace because the transmission assembly is held translationally stationary relative to the workspace by the manipulator to which the instrument is coupled.

The ability of the transmission assembly to translate relative to the instrument shaft results in a relatively complex architecture of drive and force transmission structures that make up the transmission assembly. There exists a need to provide instruments with transmission assemblies that provide the insertion degree of freedom movement of the instrument by driving relative translation between the transmission assembly and the instrument shaft while still being relatively simple and compact in its overall architecture, and/or to otherwise improve performance of instruments with such transmission assembly architectures.

### SUMMARY

Various embodiments of the present disclosure may solve one or more of the above-mentioned problems and/or may demonstrate one or more of the above-mentioned desirable features. Other features and/or advantages may become apparent from the description that follows.

In accordance with at least one embodiment of the present disclosure a medical instrument comprises an instrument shaft comprising a proximal portion and a distal portion; a movable component coupled to the distal portion of the shaft; a first actuation element coupled to the instrument shaft and a second actuation element coupled to the movable component; and a transmission assembly movably coupled to the instrument shaft. The transmission assembly comprises a plurality of drive members, including first, second, and third drive members. The first drive member comprises a rotatable drum, with the first and second actuation elements being at least partially wound around the rotatable drum and configured to drive translation of the instrument shaft relative to the transmission assembly in response to rotation of the drum. The second drive member comprises a first rotatable drive shaft, with the second actuation element being coupled to the second drive member and actuatable by rotation of the first rotatable drive shaft. The third drive member comprises a second rotatable drive shaft and a bearing coupled to the second rotatable drive shaft, with the second actuation element being routed over the bearing between the rotatable drum and the first rotatable drive shaft.

In accordance with at least one other embodiment of the present disclosure a method of manufacturing a medical instrument comprises providing an instrument shaft comprising a proximal portion and a distal portion, coupling a movable component to the distal portion of the shaft, and movably coupling a transmission assembly to the instrument shaft. The transmission assembly comprises a plurality of drive members, including first, second, and third drive members. The first drive member comprises a rotatable drum. The second drive member comprises a first rotatable drive shaft. The third drive member comprises a second rotatable drive shaft and a bearing coupled to the second rotatable drive shaft. The method further comprises coupling a first actuation element to the instrument shaft, coupling a second actuation element to the movable component, coupling the first and second actuation elements to the rotatable drum such that the first and second actuation elements are configured to drive translation of the instrument shaft relative to the transmission assembly in response to rotation of the drum, and coupling the second actuation element to the second drive member such that the second actuation element is actuatable by rotation of the first rotatable drive shaft. The method further comprises routing the second actuation element over the bearing between the rotatable drum and the first rotatable drive shaft.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1A is a schematic diagram of an embodiment of an instrument.

FIG. 1B is a schematic diagram of portions of the instrument of FIG. 1A.

FIG. 1C is a schematic cross-section of portions of the instrument of FIG. 1A, with the section taken along a longitudinal centerline of the instrument.

FIG. 2 is a perspective view of another embodiment of an instrument.

FIG. 3 is a perspective view of a transmission assembly housing of the instrument of FIG. 2.

FIG. 4 is a perspective view of an embodiment of a transmission assembly of the instrument of FIG. 2, from a first side thereof, and with a cover portion removed.

FIG. 5 is a perspective view of the transmission assembly of FIG. 4, from a second side thereof, and with the cover portion removed.

FIG. 6 is a schematic diagram of portions of the instrument of FIG. 2.

FIG. 7A is a plan view of an embodiment of an actuation transfer mechanism of the instrument of FIG. 2 in a first state.

FIG. 7B is a plan view of the actuation transfer mechanism of FIG. 7A in a second state.

FIG. 7C is a side view of the actuation transfer mechanism of FIG. 7A in the first state.

FIG. 7D is a side view of the actuation transfer mechanism of FIG. 7A in the second state.

FIG. 8 is a perspective section view of the instrument of FIG. 2 with the section taken along 7 in FIG. 5.

FIG. 9 is a perspective view of a portion of an embodiment of a shaft of the instrument of FIG. 2.

FIG. 10 is a schematic diagram of an embodiment of a teleoperable manipulator system.

FIG. 11 is a perspective view of an embodiment of an instrument manipulator.

### DETAILED DESCRIPTION

In the description below, reference is made to various types of actuation elements, such as pullable actuation elements, pushable actuation elements, and rotational actuation elements. Pullable actuation elements refers to actuation elements that comprise flexible pulling/tension members (such as cables, wires, filaments, belts, chains, straps, ropes, etc.) configured to transfer pulling (tensioning) forces while also being relatively flexible in directions perpendicular to a longitudinal dimension of the member. Pushable actuation elements refers to actuation elements that comprise relatively more rigid pushing/compression members configured to transfer pushing (compression) forces, including relatively rigid members that are capable of transmitting both pushing and pulling forces (such as rigid tubes, rods, bars, beams, etc.) and semi-flexible members that are capable of transmitting axial pushing forces (and in some cases also pulling forces) but which are more flexible in one or more lateral directions, or any combination of these. Examples of pushable actuation elements that are flexible in one or more lateral directions include push coils, cut tubes, laterally supported cables (e.g., so-called “push-cables”), thin beams or plates that are flexible along one lateral dimension, etc. Use of the term “pushable” should not be misconstrued as implying that the pushable actuation elements cannot also transfer pulling forces, as some pushable actuation elements may be capable of doing so (sometimes called push-pull members). Rotational actuation elements refers to actuation elements configured to transfer rotary motion and torque (forces urging rotary motion), such as torque tubes, screws, bars, shafts, flexible coiled wire rotary tubes, etc. In some cases, similar types of members could be described as more than one of the above noted types, depending on how the member is to be driven (e.g., a rigid rod could serve as a pushable actuation element, a rotatable actuation element, or both). In the description below, when the specific type of actuation element is not relevant, or when it is understood from the context, the actuation element may be referred to as an “actuation element” without specifying pullable, pushable, or rotatable.

In the description below, reference is made to “paying out” or “drawing in” pullable actuation elements. Paying out a pullable actuation element refers to increasing the length of a segment of the actuation element that extends between a take-off point on a drive member to which the actuation element is coupled and a coupling point at which the actuation element is operably coupled to a moveable component whose motions is driven by the actuation element. Conversely, drawing in the pullable actuation element refers to decreasing the length of the segment of the actuation element that extends between the take-off point of the drive member and the coupling point of the driven moveable component. By extension, “paid-out length” refers to the current length of the above-described segment of the pullable actuation element that extends between the take-off point and the coupling point. Furthermore, in the description below, reference is made to the “path length” for a pullable actuation element. The path length refers to the length of a hypothetical path that the above-described segment would be expected to traverse as it extends between the take-off point and the coupling point, assuming that the segment has no slack. The path length does not necessarily always equal the paid-out length, as some slack may be present resulting in the paid-out length exceeding the path length. References to “actuating” a pullable actuation element refer to causing the actuation element to be paid out or drawn in. References to “actuating” a drive member refer to causing the drive member to move (e.g., rotate) so as to perform some action, such as actuating an actuation element coupled thereto. The above-described terms and definitions are explained in further detail below, in and following the description of the various embodiments.

As noted above, in instruments for which an insertion degree of freedom occurs by a drive input at a transmission assembly of the instrument causing relative translation between the transmission assembly and the instrument shaft, the transmission assembly can become rather complicated. Some aspects of one embodiment of such a transmission assembly are described briefly below to illustrate some of the challenges that may arise in such instruments. In an embodiment of a transmission assembly, one of the drive members, also referred to hereinafter as the insertion drive member, drives translation of the instrument shaft relative to the transmission assembly to achieve the insertion degree of freedom motion of the instrument shaft and end effector. Various other drive members of such a transmission assembly, also referred to hereinafter as non-insertion drive members, can each be coupled to at least one respectively corresponding actuation element in the form of pullable actuation elements that are actuated (drawn in or paid out) to drive a degree of freedom of motion (or other function) of the instrument. The insertion drive member may comprise a drum to which multiple pullable actuation elements are coupled. The actuation elements coupled to the drum include one actuation element coupled to a proximal portion of the instrument shaft to drive relative translation of the shaft and transmission assembly in one direction, and also at least some of the actuation elements that are coupled to the non-insertion drive members (in some cases, all of the actuation elements that are coupled to the non-insertion drive members). The insertion drive member and the non-insertion drive members are configured such that rotation of the insertion drive member actuates (pays out or draws in) all of the actuation elements coupled thereto independently of rotation of the non-insertion drive members, thereby causing relative translation between the shaft and transmission assembly but without consequent actuation of the movable components (e.g., end effector, wrist, etc.) coupled to the non-insertion drive members. Individual actuation elements coupled to the non-insertion drive members can also be actuated independently of the insertion drive member to cause actuation of a movable component by rotation of a corresponding one of the non-insertion drive members. In such a transmission assembly, there can be numerous actuation element segments extending between the insertion drive member and the non-insertion drive members as well as segments extending between the various drive members and the instrument shaft. It can be challenging to arrange the drive members and the actuation elements of the transmission assembly in an orderly manner that allows the cables and drive members to function as intended without tangling or otherwise interfering with one another. This can be particularly challenging when trying to design a more compact transmission assembly or when trying to include a large number of drive members or other components in the transmission assembly, as generally speaking the smaller the transmission assembly is, or the more drive members or other components are included therein, the more likely it is that interference occurs with the actuation elements routed through the transmission assembly.

Another challenge for instruments in which the transmission assembly drives the insertion degree of freedom pertains to maintaining pressurization within a workspace. In some use cases, such as in some types of medical application for example, a workspace may be pressurized to insufflate the workspace by flowing a fluid (e.g., air or another gas) into the workspace. Insufflation may provide various benefits, such as, for example, increasing an amount of space within the workspace (e.g., by expanding a body cavity of the patient). To prevent fluid from escaping the pressurized workspace when an instrument shaft is inserted therein, an insufflation seal is generally provided between the instrument shaft and a cannula through which the instrument shaft is inserted into the workspace. However, in instruments in which the transmission assembly and the shaft translate relative to one another, actuation elements may be routed along an exterior of the shaft for some portions of the shaft, and if this transition zone is located proximal of the insufflation seal of the cannula then fluids may escape through the transition zone. Moreover, attempting to provide the transition zone distal of the insufflation seal of the cannula may limit a range of motion of the instrument. Thus, it can be difficult to adequately seal such instruments while also providing desired range of motion

Another challenge pertains to slack that can arise in pullable actuation elements when tension is not actively applied to the actuation element, such as, for example, may occur when the instrument is not mounted to a manipulator in the case of a teleoperated instrument. If sufficient slack is developed in an actuation element, the actuation element, such as a cable, can derail from a handling component, such as a pulley or the drive members. Slack can occur as a result of movable components controlled by the actuation elements being moved while the drive members are free to rotate, such as in the unmounted state of the instrument relative to the manipulator. By way of example, a moveable component of an instrument that is coupled to actuation elements not being actively held in tension (such as due to being mounted to a manipulator and/or otherwise having drive members clocked to a degree of rotation that tightens the actuation elements) may experience an external force, for example because the instrument has accidentally been bumped against something. Because the drive members may be free to rotate (e.g., when in an unmounted state from a manipulator system or otherwise in an unlocked state), the force applied to the moveable component may cause it to move. Such movement in turn causes one of the actuation elements coupled thereto to pay out, while generating slack in the other actuation element coupled thereto. More specifically, as the moveable component moves the path length for a first actuation element increases and the moveable component pulls against the first actuation element, and because the corresponding drive member is free to rotate the first actuation element is allowed to pay out in response to the pulling. However, the same motion of the moveable component decreases the path length for a second actuation element of the pair coupled to operate the moveable component. The paid-out length of the second actuation element does not change, and therefore as the path length decreases a difference between the paid-out length of the second actuation element and the path length for the actuation element increases, resulting in slack in the second actuation element. If enough slack develops, the second actuation element may be susceptible to derailing from a handling device.

To address some of the issues noted above and otherwise improve remotely controlled instruments, some embodiments disclosed herein provide bearing mechanisms on a drive shaft of one or more of the non-insertion drive members. The bearing mechanism provided on one drive member is configured to redirect an actuation element that is actuatable by (coupled to) another drive member as that actuation element extends between the insertion drive member and the drive member to which it is coupled. In some embodiments, the bearing mechanism may be coupled to the drive shaft and rotatable about the drive shaft such that the bearing can move with the actuation element as it is actuated, thus reducing the amount of friction that resists the actuation of the actuation element. Bearing mechanisms such as those described herein allow for the drive members to be positioned in arrangements that otherwise may not be tenable. For example, if a first non-insertion drive member is positioned between the insertion drive member and a second non-insertion drive member, then the first non-insertion drive member may block or otherwise interfere with the actuation element that is actuated by the second non-insertion drive member as that actuation element extends between the insertion drive member and the second non-insertion drive member. But by providing a bearing mechanism on the shaft of the first non-insertion drive member, the actuation element of the second non-insertion drive member can be redirected around the first non-insertion drive member by the bearing mechanism, thus allowing the actuation element to be routed between the insertion drive member and the second non-insertion drive member despite the first non-insertion drive member being positioned therebetween. Accordingly, using the bearing mechanisms described herein expands the possibilities for how the drive members can be arranged relative to one another, including allowing the drive members to be positioned closer together than may otherwise be feasible. This may allow for the transmission assembly to be made more compact or to fit additional components within a given size of the transmission assembly. Moreover, because the bearing mechanism is coupled to the drive shaft of a drive member in some embodiments, as opposed to being provided separate from the drive members for example, the need to provide an additional component in the transmission assembly to support the bearing mechanism can be avoided. In addition, because the bearing mechanism is coupled to the drive shaft of the drive member in some embodiments, the bearing mechanism may fit within a footprint that would already be occupied by the drive member, thus avoiding the need to allocate additional space within the transmission assembly for the bearing mechanism and an associated supporting component.

Furthermore, to address some of the issues noted above and otherwise improve remotely controlled instruments, some embodiments disclosed herein may provide a seal between an interior surface of a housing of the transmission assembly and an exterior surface of the shaft. In particular, the transmission assembly may comprise a proximal portion that houses the drive members and a distal portion that extends distally from the proximal portion and encircles the instrument shaft. The seal may be positioned at a proximal end of the distal portion of the housing. The seal may conform to the grooves in the shaft to seal the shaft relative to an inner surface of the housing. An outer surface of the housing may then be sealed relative to a cannula through which the instrument shaft is inserted using another seal. Thus, the two seals, the housing, and the cannula can cooperate together to reduce or prevent the escape of fluids (e.g., insufflation fluid and/or other fluid from a body cavity in which the instrument is inserted) via the shaft.

To further address some of the issues noted above and otherwise improve remotely controlled instruments, some embodiments disclosed herein may provide biasing devices for at least some of the drive members to prevent or take up slack that might otherwise develop in the actuation elements driven by those drive members, which may occur, for example, in an unmounted state of the instrument to the manipulator arm or in a state of the instrument in which the drive member is not otherwise locked in a particular position by an external force. Each biasing device comprises one or more biasing elements. A given biasing element may be coupled to one of the drive members so as to bias the drive shaft of the drive member toward rotation in a direction that draws in the actuation element coupled to the drive member. In other words, the biasing element applies a biasing force to the drive member which results in a torque urging the drive member to rotate to draw in the actuation element. In a resting state, tension in the actuation element may oppose the biasing force and may be equal in magnitude thereto, resulting in the net torque applied to the drive member being zero, and thus the drive member does not rotate. However, if the opposing tension force is reduced relative to the biasing force (e.g., due to slack beginning to develop in the actuation element), the net torque applied to the drive member will now be non-zero and the drive member with thus rotate in the direction that draws in the actuation element. The drawing in of the actuation element counteracts the reduction in tension, preventing or mitigating the development of the slack. In some embodiments, the one or more biasing elements of the biasing device comprises torsion springs.

In some embodiments, a given biasing device may comprise a pair of the above-described biasing elements, and these biasing elements may be coupled, respectively, to a pair of the drive members that actuate a pair of actuation elements to drive motion of the same movable component along opposite directions of a given degree of freedom of motion. Because the actuation elements are linked together (via the movable component to which both actuation elements are coupled), in a resting state the biasing force applied directly to one drive member by a biasing element is also applied indirectly to the other drive member via the actuation elements, resulting in opposing forces being applied to each of the drive members by the biasing elements. In a resting state, these opposing forces applied to the drive members by the biasing device are equal and cancel one another out, resulting in an equilibrium in which zero net torque is applied to the drive members and the drive members do not move (similarly, zero net force and/or net torque is applied to the moveable component, and the moveable component thus does not move). However, if an external force is applied to the movable component, this equilibrium may be upset. For example, if an external force causes the first drive member of the pair to pay out a first actuation element coupled to the first drive member, this reduces (or eliminates) the tension in the second actuation element coupled to the second drive member. As a result of the reduced tension in the second actuation element, the biasing force being applied directly to the second drive member is no longer opposed (or is opposed by a weaker opposing force), and therefore a non-zero net torque is now applied by the biasing device to the second drive member. This non-zero net torque urges the second drive member to rotate and draw in the second actuation element of the pair. In other words, the biasing device is configured to, automatically and passively, cause one drive member of the pair to draw in the actuation element coupled thereto in response to the other drive member being forced to pay out of the actuation element coupled thereto, and vice versa. Consequently, any slack that might have otherwise been created as a result of the external force is avoided or taken up.

In some examples in which a given biasing device comprises a pair of biasing elements, the biasing elements are directly connected together by a linkage. In other examples in which a given biasing device comprises a pair of biasing elements, the biasing elements are separate parts which are not directly connected together.

Turning now to the figures, various embodiments will be described in greater detail.

### First Instrument Embodiment

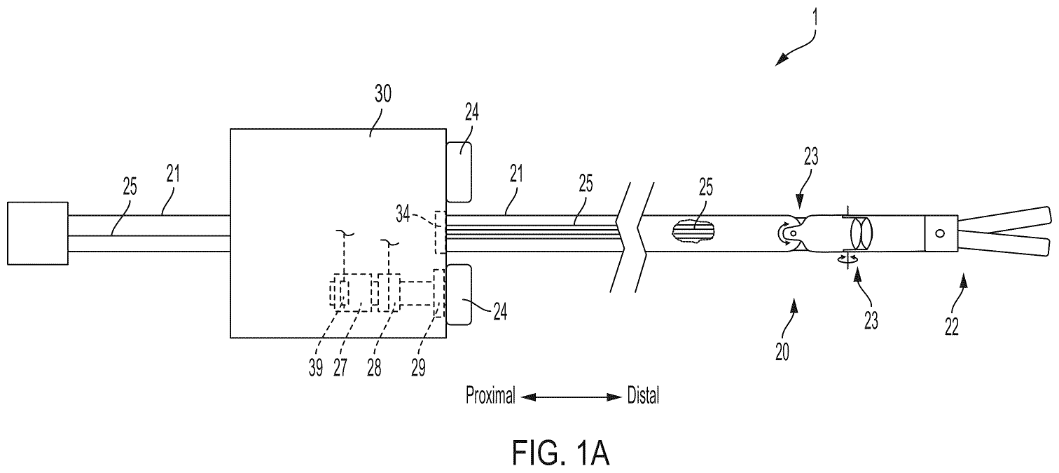

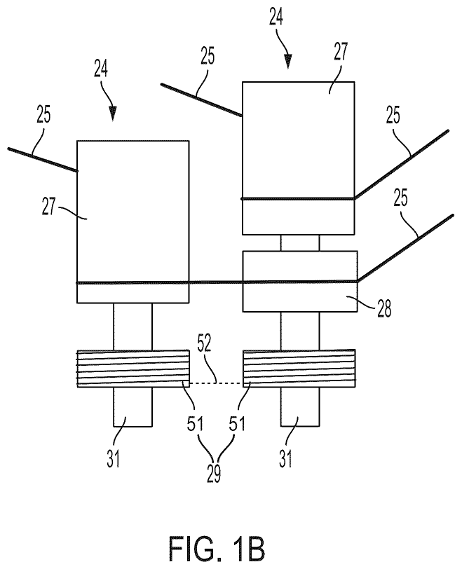

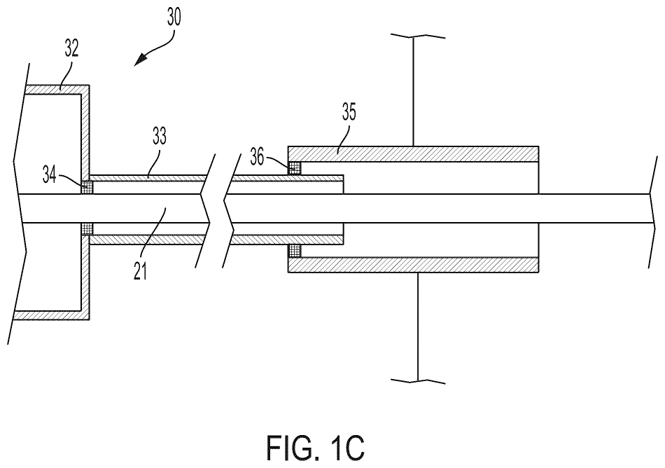

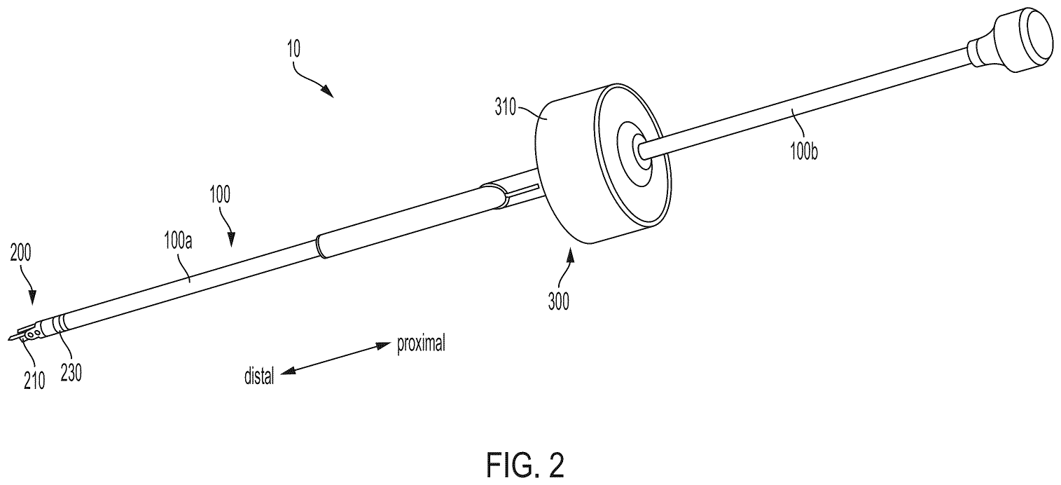

FIGS. 1A-1C illustrates an embodiment of an instrument 1. FIGS. 2-9 illustrates various views of another embodiment of an instrument 10. The instrument 10 may be used as the instrument 1. In some embodiments, it is contemplated that either of the instruments 1 or 10 is used with and controlled via a computer-controlled teleoperated system, such as the system 1000 described below in relation to FIG. 10. For example, the instruments 1 or 10 may be used as the instrument 1002. While various embodiments described herein contemplate an instrument configured to be mounted to a manipulator system of a computer-controlled teleoperated system, those having ordinary skill in the art would appreciate that a manually-operated instrument is also contemplated, in which case various drive members can be configured to be operated via manual drive inputs, such as wheels, buttons, and the like.

In some embodiments, the instrument 1 or 10 is a medical instrument, which may be used to perform medical procedures, such as, for example, surgical, diagnostic, or therapeutic procedures. Medical instruments may include a variety of instruments used to perform medical procedures, such as therapeutic instruments, diagnostic instruments, surgical instruments, and/or imaging instruments. In some examples, the medical instruments may be inserted into a patient through a natural orifice or an incision (including through a port or other guide inserted in the incision). Such instruments that are remotely controlled may be particularly useful, for example, in performing minimally invasive surgical procedures. A minimally invasive medical procedure may be designed to reduce the amount of tissue that is damaged during the procedure, for example by decreasing the number and/or size of incisions through which medical instruments are inserted. In other embodiments, the instrument 1 or 10 may be a non-medical instrument, such as an industrial instrument used for remote inspection or other remote procedures.

As shown in FIG. 1A, an embodiment of an instrument 1 comprises a shaft 21 supporting an end effector 22 at a distal end of the shaft 21 (proximal and distal directions referenced herein are illustrated in FIG. 1A). The end effector 22 is configured to perform one or more functions. The instrument 1 also comprises one or more movable components 20 coupled to a distal end portion of the shaft 21. The one or more movable components 20 may comprise one or more components of the end effector 22 that are drivable to move (e.g., a jaw member, a translating cutting component, etc.) and/or one or more articulable structures 23 (e.g., forming a wrist mechanism). The instrument 1 also comprises a force transmission assembly 30, which is movably coupled to the shaft 21 proximal of the movable components 20 (the shaft 21 and force transmission assembly 30 move relative to one another, as described below, and thus the coupling location of the transmission assembly 30 to the shaft 21 varies, but the transmission assembly 30 remains positioned generally proximal of the movable component(s) 20 of the instrument 1).

The end effector 22 is illustrated as having a jaw mechanism in FIG. 1A, but it should be understood that any other type of end effector could be used. Some non-limiting examples of end effectors 22 include staplers, forceps, vessel sealers, imaging devices (e.g., an endoscope tip), sensing devices (pressure, temperature, etc.) scissors, electrosurgical devices (e.g., monopolar or bipolar electrosurgical devices), other flux delivery devices (irrigation, suction, etc.), and so on. Functions performed by end effector 22 may include, for example, grasping, cutting, stapling, electrosurgical functions, illuminating, image capture, fluid delivery and/or evacuation, etc., as would be familiar to those of ordinary skill in the art. In some embodiments, one or more functions of the end effector 22 may involve motion of a movable component that is mechanically driven. In some embodiments, some functions of the end effector 22 may be driven non-mechanically, for example, by supplying electrical power or other functional flows (e.g., vacuum suction, light, etc.) to the end effector 22. Some functions of the end effector 22 may involve a combination of mechanical and non-mechanical driving inputs, such as an electrosurgical function which may comprise a mechanically driven operation of closing of a jaw member to grasp an object and an electrically driven operation of sealing or cutting the grasped object. In some embodiments, the end effector 22 does not necessarily comprise any movable components that are mechanically driven—by way of nonlimiting example, the end effector 22 may comprise an endoscope tip with an imaging device or a monopolar electrosurgical tip member.

The force transmission assembly 30 comprises drive members 24 configured to receive input driving forces, with the driving forces controlling degrees of freedom of motion of the instrument 1. The drive members 24 convert and transfer the driving forces to actuation elements 25, such as pullable actuation elements and/or push-pull actuation elements, to drive motion of the movable components 20 or other parts of the instrument 1. Some of the actuation elements 25 may extend distally from the force transmission assembly 30 through and/or along the shaft 21 and are coupled to the movable components 20. In some embodiments, the distally extending actuation elements 25 may pass along an exterior of the shaft 21 for a portion thereof and then may pass through an interior of the shaft 21 along another portion thereof, as illustrated in FIG. 1A. In some embodiments, one or more actuation elements 25 may also extend proximally from the force transmission assembly 30. In various embodiments, the force transmission assembly is mountable to a manipulator of a computer-controlled, teleoperated system (such as the system 1000 described in further detail below), and the input driving forces are provided through the manipulator interface (as also describe in further detail below).

One of the drive members 24 (also referred to as insertion drive member 24) is configured to drive an insertion degree of freedom of motion of the instrument (i.e., relative translation between the instrument shaft 21 and the force transmission assembly 30) by actuating two or more of the actuation elements 25 coupled thereto. In particular, one of the proximally extending actuation elements 25 is coupled with the insertion drive member 24 and a proximal portion of the shaft 21 such that the insertion drive member 24 can drive translation of the shaft 21 in a distal direction relative to the transmission assembly 30 by drawing in this proximally extending actuation element 25. In addition, one or more of the distally extending actuation elements 25 is coupled with the insertion drive member 24 and with a part the instrument that is distal of the force transmission assembly 30, such as a movable component 20, such that the insertion drive member 24 can drive translation of the shaft 21 in a proximal direction relative to the transmission assembly 30 by drawing in these distally extending actuation element 25. When the force transmission assembly 30 is in a fixed position, such as mounted to a manipulator or otherwise held in a fixed position, the above-noted relative translation results in translation of the shaft 21 relative to the fixed position at which the force transmission assembly 30 is coupled, which can be used for insertion or withdrawal of the end effector 22 relative to the workspace.

One or more other drive members 24 (also referred to as non-insertion drive members 24) are configured to drive additional degrees of freedom of motion by actuating one or more respectively corresponding actuation elements 25 coupled thereto. These actuation elements 25 may be coupled to the movable components 20 to drive motion thereof.

In some embodiments the one or more movable components 20 of the instrument 1 include one or more articulable structures 23 (such as jointed links, flexible portions of a shaft, etc.). Each articulable structure 23 has at least one corresponding degree of freedom of motion, which is driven by one or more actuation elements 25 coupled to the articulable structure 23. The articulable structure 23 can be used to couple the end effector 22 to the shaft 21 to allow for relative motion between the end effector 22 (or some other component) and the shaft 21, thereby allowing the pose of the end effector 22 to be changed. Such placement for an articulable structure 23 is nonlimiting and articulable structures 23 can be used along the instrument shaft 21 to provide differing poses of portions of the shaft relative to other portions of the shaft 21, as those having ordinary skill in the art would be familiar with. In some embodiments, the articulable structures 23 provides differing degrees of freedom of motion. In some embodiments, multiple articulable structures 23 having differing degrees of freedom of motion are connected in series to form a wrist mechanism that couples the end effector 22 to the shaft 21 and to enable the end effector 22 to move with two or more degrees of freedom of motion (e.g., yaw, pitch, or combinations thereof) relative to the shaft 21. In some embodiments, the end effector 22 is coupled directly to the shaft 21 without an intervening articulable structure 23. References herein to an end effector being coupled to or supported by a shaft should be understood as broadly including both direct coupling and indirect coupling (e.g., via an articulable structure 23 or other intervening component), unless otherwise indicated or implied by the context. Although two articulable structures 23 comprising rotating joints are illustrated in FIG. 1A, it should be understood that this is not limiting and any number or type of articulable structure 23 could be used instead, including zero, one, three, or more articulable structures 23.

Moreover, in some embodiments, the one or more movable components 20 of the instrument 1 comprise movable components of the end effector 22 (in addition to, or in lieu of the articulable structures 23). In such embodiments, the movable components of the end effector 22 may be mechanically driven by driving forces transmitted to the end effector 22 from the drive members 24 via actuation elements 25. Examples of such movable components of an end effector 22 may include a translatable blade, a pivotable jaw member of a jaw mechanism, a translatable staple firing shuttle of a stapler, etc. Such movable components of the end effector 22, when present, are instances of the above-noted movable components 20, and their motion may be considered a degree of freedom of motion of the instrument 1.

The instrument 1 may also have additional degrees of freedom of motion. For example, in some embodiments the shaft 21 may be rotatable about its longitudinal axis, relative to the workspace, by rotation of the entire instrument 1 about the longitudinal axis. This may occur, for example, by rotation of an instrument holder to which the instrument 10 is mounted relative to a support that the instrument holder is coupled with (and hence rotation of the instrument 1 as a whole relative to the support). Such rotation of the instrument shaft 21 may be referred to as a roll degree of freedom of motion.

In some embodiments, one or more of the distally extending actuation elements 25 that is coupled to one of the non-insertion drive members 24 is also coupled to the insertion drive member 24—in other words, at least one of the actuation elements 25 is coupled to and actuatable by both the insertion drive member 24 and a non-insertion drive member 24. This non-insertion drive member 24 may comprise an actuation transfer mechanism 27 that allows both the insertion drive member 24 and the non-insertion drive member 24 to actuate the same actuation element 25 independently of one another.

In some embodiments, the instrument 1 may also comprise any combination of one or more of: a bearing mechanism 28, a biasing device 29, and/or a seal 34, as indicated in FIG. 1A and as shown in greater detail in FIGS. 1B and 1C. In FIG. 1A, all three of the aforementioned parts are illustrated for ease of discussion, but it should be understood that embodiments with different combinations of these components are contemplated herein, including: embodiments with a bearing mechanism 28 but no biasing device 29 or seal 34, embodiments with a biasing device 29 but no bearing mechanism 28 or seal 34, embodiments with an seal 34 but no bearing mechanism 28 or biasing device 29, embodiments with any combination of two of the aforementioned parts, and embodiment with all of the aforementioned parts.

In embodiments that comprise the bearing mechanism 28, the bearing mechanism 28 may be provided on a drive shaft 31 of one or more of the non-insertion drive members 24, as shown in FIG. 1B. The bearing mechanism 28 provided on one drive member 24 is configured to redirect an actuation element 25 that is actuatable by (coupled to) another drive member 24 as that actuation element 25 extends between the insertion drive member 24 and the drive member 24 to which the actuation element 25 is coupled. In some embodiments, the bearing mechanism 28 may be coupled to the drive shaft 31 and rotatable about the drive shaft 31 such that the bearing mechanism 28 can move with the actuation element 25 as it is actuated, thus reducing the amount of friction that resists the actuation of the actuation element 25.

In embodiments that comprise the biasing device 29, a biasing device 29 may be provided for at least one of the non-insertion drive members 24. As described above, the biasing device 29 comprises at least one biasing element 51 coupled to the drive member 24 and configured to bias the drive member 24 toward rotation in a direction that draws in the actuation element 25 coupled to the drive member 24. That is, the biasing element 51 biases the drive member 24 to generate tension in the actuation elements 25 coupled thereto. The biasing element 51 may be, for example, a torsion spring or any other type of biasing element that can supply the biasing force. In FIG. 1B, two biasing elements 51 are illustrated, but any number of biasing elements 51 may be provided, including one or more.

More specifically, in some embodiments a biasing device 29 comprises a pair of two biasing elements 51 and the biasing device 29 may be provided for at least one corresponding pair of non-insertion drive members 24, as shown in FIG. 1B, with one of the biasing elements 51 being coupled to one drive member 24 of the pair and the other biasing element 51 being coupled to the other drive member 24 of the pair. The biasing device 29 is configured to prevent or take up slack that might otherwise develop in the actuation elements 25 driven by those drive members 24, which may occur, for example, in an unmounted state of the instrument 1 to the manipulator arm or in a state of the instrument 1 in which the drive members 24 are not otherwise locked in a particular position by an external force. In some embodiments, the pair of the drive members 24 to which the biasing device 29 is coupled drive motion of the same movable component 20 along opposite directions of a given degree of freedom of motion. The biasing device 29 may bias the drive members 24 to rotate in respective directions that draw in the respective actuation elements 25 coupled thereto. Consequently, if one of the drive members 24 of the pair is caused by an external force to rotate in a direction that pays out the actuation element 25 coupled thereto, then the biasing device 29 will generate a non-zero net torque on the other drive member 24 that urges the other drive member 24 to rotate in a direction that draws in the actuation element 25 coupled thereto, thus taking up any slack that may have otherwise developed due to the paying out of the first actuation element 25. In some embodiments, the biasing elements 51 of the biasing device 29 may comprise one or more springs, such as torsion springs, for example. The biasing elements 51 of the biasing device 29 may also be coupled (directly or indirectly) to the drive shafts 31 of the drive members 24, as shown in FIG. 1B.

In some embodiments in which the biasing device 29 comprises a pair of biasing elements 51, these biasing elements 51 are coupled together by a linkage 52. More specifically, a first end of each biasing element 51 is coupled to the drive member 24 while a second end of each biasing element 51 is coupled to the linkage 52. In this way, the second end of one biasing element 51 is anchored relative to the other biasing element 51, and vice versa. In some embodiments, the linkage 52 is integrally coupled to the biasing elements 51; in other words, the biasing elements 51 and linkage 52 are all part of a single unitary body (e.g., a single piece of metal). The linkage 52 may beneficially allow the biasing device 29 to be formed as a single physical part, which can reduce the number of parts needed for the instrument 1 and thus simplify and reduce the cost of manufacture as compared to if the biasing device 29 comprises multiple distinct pieces.

In other embodiments, the linkage 52 is omitted and the biasing elements 51 are physically separate parts which are not directly coupled together. In such examples, the second ends of the biasing elements 51 may be anchored to some other portion of the transmission assembly 30, such as a housing or other support structure which supports the drive member 24. In some embodiments, retention elements 39 may be used in addition to or in lieu of the drive members 24 to help to prevent derailing of actuation elements 25 from handling devices, such as actuation transfer mechanisms 27, pulleys, drums, capstans, etc. Such retention elements 39 may comprise pieces of material that are disposed adjacent to a portion of the handling device so as to cover an opening of the handling device, thus preventing an actuation element 25 routed between the retention element 39 and the handling device from derailing.

In embodiments that comprise the seal 34, the seal 34 may be positioned around the shaft 21 at an opening in a housing of the transmission assembly 30 through which the shaft 21 passes, creating a seal between the shaft 21 and the housing. In some embodiments, the housing of the transmission assembly 30 may comprise a proximal portion 32, which houses the drive members 24, and a distal portion 33 which extends from the proximal portion 32 and surrounds the instrument shaft 21, and the seal 34 may be placed between an inner surface of the distal portion 33 and an exterior surface of the shaft 21, as shown in FIG. 1C. The seal 34 may prevent fluids, such as insufflation gasses, from passing into the proximal portion 32. The seal 34 may conform to grooves in the shaft 21 to allow for a better seal, which in some embodiments may be gas tight. During usage, the distal portion 33 may be inserted into a cannula 35 or other entry guide structure, and an outer surface of the distal portion 33 may be sealed relative to a cannula 35, e.g., by another seal 36. In some embodiments, the seal 34 is located proximal of an opening in the shaft 21 at which actuation elements 25 enter and exit an interior of the shaft 21, thus allowing for the seal 34 to also prevent leakage of fluids via that opening in the shaft 21.

### Second Instrument Embodiment

Turning now to FIGS. 2-9, another embodiment of an instrument 10 is described below. The instrument 10 may be used as the instrument 1. The descriptions above of parts of the instrument 1 are applicable mutatis mutandis to the similar parts of the instrument 10.

As shown in FIG. 2, an embodiment of an instrument 10 comprises a shaft 100 supporting an end effector 210 at a distal end of the shaft 100 (proximal and distal directions referenced herein are illustrated in FIG. 2). The end effector 210 is configured to perform one or more functions, similar to the end effector 22 as described above. The instrument 10 also comprises one or more movable components 200, similar to the movable components 20 described above, coupled to a distal end portion of the shaft 100. The one or more movable components 200 may comprise one or more components of the end effector 210 (e.g., a jaw member of a jaw assembly, a translating component such as a cutting component, etc.) and/or one or more articulable structures 230 coupling the end effector 210 (or some other component of the instrument 10) to the shaft 100 and/or provided along the shaft 100 to impart articulation to the shaft 100. The instrument 10 also comprises a force transmission assembly 300, similar to the force transmission assembly 30 described above, which is movably coupled to the shaft 100 proximal of the movable components 200 (the shaft 100 and force transmission assembly 300 move relative to one another, as described below, and thus the coupling location of the transmission assembly 300 to the shaft 100 varies, but the transmission assembly 300 remains positioned generally proximal of the movable component(s) 200 of the instrument 10).

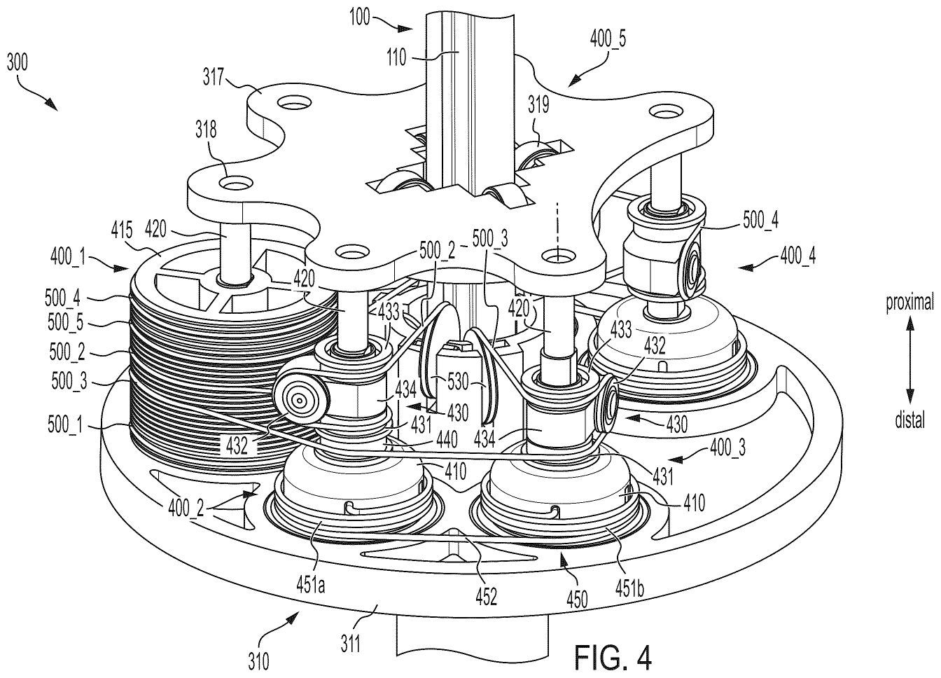

The force transmission assembly 300 may be used as the force transmission assembly 30, and comprises drive members 400 (described in greater detail below in relation FIG. 4) configured to receive input driving forces, with the driving forces controlling degrees of freedom of motion of the instrument 10. The drive members 400 may be used as the drive members 24 described above. The drive members 400 convert and transfer the driving forces to actuation elements 500 (e.g., actuation elements 500_1 to 500_5, see FIG. 4), such as pullable actuation elements and/or pushable actuation elements, as described above. The actuation elements 500 extend through and/or along the shaft 100 and are coupled to the movable components 200 and/or other parts of the instrument 10 to transmit the driving forces so as to drive various degrees of freedom of motion of the instrument 10, which may include degrees of freedom of motion of the movable components 200 as well as additional degrees of freedom of motion. In various embodiments, the force transmission assembly 300 is mountable to a manipulator of a computer-controlled, teleoperated system (such as the system 1000 described in further detail below), and the input driving forces are provided through the manipulator interface (as also describe in further detail below).

As noted above, in some embodiments the instrument comprises articulable structures 230 (such as jointed links, flexible portions of a shaft, etc.), and each articulable structure 230 has at least one corresponding degree of freedom of motion, which is driven by one or more actuation elements coupled to the articulable structure 230. Components of the force transmission assembly 300 are described below in greater detail with reference to FIGS. 2-9. The force transmission assembly 300 may be used as the force transmission assembly 30 described above. For organizational purposes and to assist in readability, the description includes subheadings for the various components that will be described. However, it should be understood that while in some embodiments, the various components are used together, other embodiments of force transmission assemblies may not necessarily include all the components discussed. In particular, embodiments disclosed herein may comprise a bearing mechanism 440 (which may be used as the bearing mechanism 28), a biasing device 450 (which may be used as the biasing device 29), and/or a seal 550 (which may be used as the seal 34), and these components can be used individually or in any combination. More specifically, some embodiments of the force transmission assembly include all three of the bearing mechanisms 440, biasing devices 450, and insufflation seal 550 as illustrated in FIGS. 2-9, other embodiments of the force transmission assembly include the bearing mechanisms 440 but not the biasing devices 450 or insufflation seal 550, other embodiments of the force transmission assembly 300 include the biasing devices 450 but not the bearing mechanisms 440 or insufflation seal 550, other embodiments of the force transmission assembly include the insufflation seal 550 but not the bearing mechanisms 440 or biasing devices 450, and other embodiments of the force transmission assembly include any combination of two of the bearing mechanisms 440, biasing devices 450, and insufflation seal 550.

### Force Transmission Assembly Housing

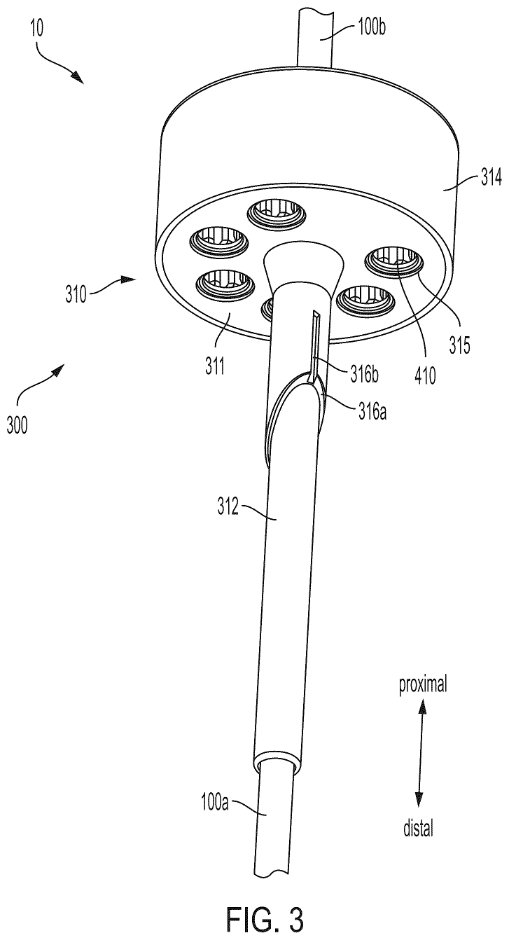

As shown in FIGS. 2 and 3, the force transmission assembly 300 comprises a housing 310. The housing 310 comprises a chassis 311, a sleeve 312 extending distally from the chassis 311, and a cover portion 314. The chassis 311 serves as a platform or a base that supports other components of the force transmission assembly 300, such as the drive members 400 described in further detail below. The chassis 311 and the cover portion 314 cooperate together to define a chamber in the housing 310 within which the drive members 400 and other components of the force transmission assembly 300 are housed. The chamber defined by the chassis 311 and the housing 310 may be used as the proximal portion 32 described above. The sleeve 312 may be used as the distal portion 33 described above.

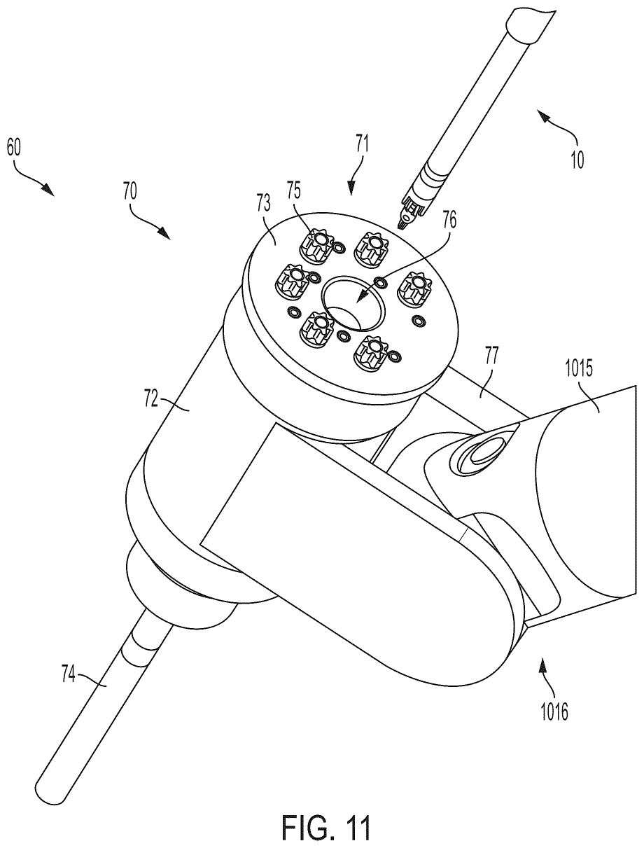

In the embodiment of FIGS. 2-9, the chassis 311 also provides an interface for coupling the instrument 10 to a manipulator. Specifically, a distal face of the chassis 311, visible in FIG. 3, forms an interface for the instrument 10 that is mountable to a complementary interface of the manipulator (e.g., interface 73, described below with respect to FIG. 11). The chassis 311 may engage either directly with the manipulator, or the chassis 311 may engage indirectly with the manipulator via an intermediary device, such as a sterile adaptor that is interposed between the manipulator and the instrument 10 to provide a sterile barrier. As shown in FIG. 3, the distal face of the chassis 311 comprises openings 315 through which drive inputs 410 of the drive members (visible in FIGS. 4, 5 and 8) are accessible from an exterior of the chassis 311, for example from the distal side of the chassis 311 in the depicted embodiment. Corresponding drive outputs of the manipulator to which the instrument 10 is mounted (e.g., drive outputs 75, described below with respect to FIG. 11) may thus interact with the drive inputs 410 via the openings 315 to transfer driving forces to the drive inputs 410. The drive outputs of the manipulator may engage directly with the drive inputs 410, or they may be engaged indirectly via an intermediary, such as intermediate couplers of the sterile adaptor. The drive inputs 410 and the drive outputs (or intermediate couplers) may have complementary engaging surfaces, such as engaging surfaces with complementary groves and protrusions or other complementary keying features, such that when the drive inputs 410 are engaged with the drive outputs or intermediate couplers they are constrained to rotate together. In FIG. 3, the drive inputs 410 are shown as having receptacles into which complementary drive outputs or intermediate couplers are received, but in other embodiments, the drive inputs 410 may comprise protrusions configured to be received within complementary receptacles of the drive outputs or intermediate couplers, or any of a variety of other mating engagements to allow the drive inputs to engage with the corresponding drive outputs or intermediate couplers. In FIG. 3, the drive inputs 410 are shown as being wholly contained within the housing 310, but in other embodiments the drive inputs 410 may extend distally from the chassis 311 through the openings 315.

A sleeve 312 of the housing 310 surrounds the instrument shaft 100 and extends distally from the chassis 311. The sleeve 312 is configured to be insertable into a passage of a manipulator when the instrument 10 is mounted to the manipulator. The sleeve 312 facilities sealing of the workspace to prevent escape of pressurized fluid. The sleeve 312 may also comprise alignment features 316 to aid in alignment of the instrument 10 relative to the manipulator as the instrument 10 is mounted to the manipulator. For example, FIG. 3 illustrates two alignment features 316, namely first alignment feature 316a and second alignment feature 316b. The first alignment feature 316a comprises an angled surface portion forming a shoulder or series of shoulders extending circumferentially around the sleeve 312 and configured to engage with complementary surfaces of the manipulator as the sleeve 312 is inserted into the passage of the manipulator to provide progressively increasing alignment between the instrument 10 and the instrument holder the further the instrument 10 is inserted into the passage. The second alignment feature 316b may comprise one or more straight notches extending proximally from an apex of the first alignment feature 316a and generally parallel to a longitudinal axis of the instrument shaft 100. The second alignment feature 316b interacts with a complementary feature of the manipulator once the instrument 10 has been inserted sufficiently far into the passage. The first alignment feature 316a is configured such that they will bring the instrument 10 into an aligned orientation by the time the instrument 10 has advanced sufficiently into the passage for the second alignment features 316b to be engaged. The second alignment feature 316b then holds the instrument 10 in the aligned orientation as the instrument 10 continues to be advanced into the passage until a mounted position is reached. The initial alignment forced by the first alignment feature 316a may bring the drive inputs 410 into alignment with the corresponding drive outputs of the manipulator prior to coupling thereof, and then the continued advancement of the instrument thereafter while the second alignment feature 316b maintains the aligned orientation may enable the coupling between the drive inputs 410 and drive outputs to be completed.

In the illustrated embodiment, the sleeve 312 and the chassis 311 are integrally coupled together, or in other words they are both part of the same monolithic body. In other embodiments, the sleeve 312 and the chassis 311 are separately formed parts that are coupled together, for example, by mechanical fasteners, friction fitting, adhesive, welding, or other known joining techniques.

### Drive Members—Overview

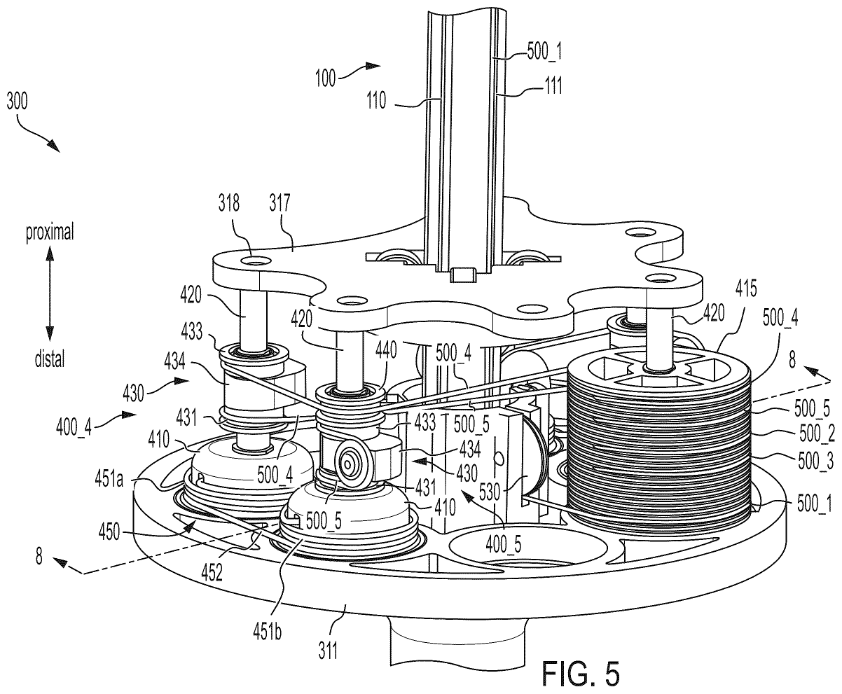

Turning now to FIGS. 4 and 5, embodiments of drive members 400 and other related components of the transmission assembly 300 will be described in greater detail. FIGS. 4 and 5 comprise perspective views from opposite sides of the transmission assembly 300, with the cover portion 314 removed to show the drive members 400.

As shown in FIGS. 4 and 5, the transmission assembly 300 comprises a plurality of drive members 400. The drive members 400 each actuate (i.e., pay out or draw in) one or more actuation elements 500, which drives a corresponding degree of freedom of motion of the instrument 10. While the drive members 400 depicted and discussed with regarding the embodiment of the force transmission assembly 300 are operably coupled to actuate pullable type actuation elements (e.g., a cable, wire, filament, etc.), those having ordinary skill in the art would appreciate that a force transmission assembly in accordance with the present disclosure could include additional drive members operably coupled to actuate pushable and/or rotatable type actuation elements. In the illustrated embodiment, the transmission assembly 300 comprises at least two drive members 400, with a first drive member 400_1 being configured to drive an insertion degree of freedom of motion and one or more other drive members 400_2 to 400_N (where N is any integer equal or greater than two) being configured to drive at least one other degree of freedom of motion of the instrument 10. The drive member 400_1 may also be referred to herein as an “insertion drive member,” and the other drive members 400_2 to 400_N may also be referred to as “non-insertion drive members.” In the illustrated embodiment, five drive members 400 are provided, but in other embodiments other numbers of drive members 400 can be used.

As noted above, each drive member 400 drives a corresponding degree of freedom of motion of the instrument 10 by paying out or drawing in at least one corresponding actuation element 500. In particular, as shown in FIGS. 4 and 5, each drive member 400 comprises a drive shaft 420 about which the drive member 400 is rotatable relative to the chassis 311, with rotation of the drive member 400 about its drive shaft 420 causing the corresponding actuation element(s) 500 to pay out or draw in, depending on the direction of rotation. Paying out or drawing in one of the actuation elements 500 may also be referred to herein generically as “actuating” the actuation element 500 when the particular direction of motion (i.e., paying out or drawing in) is not specified. Similarly, causing the drive shaft 420 of a drive member 400 to rotate may also be referred to herein as “actuating” the drive element 400. Thus, using this nomenclature, a given actuation element 500 may be actuated by actuating a corresponding drive member 400.

As shown in FIGS. 3-5, each drive member 400 also comprises a drive input 410 coupled to the drive shaft 420 such that the drive shaft 420 is constrained to rotate with the drive input 410. As noted above, in a mounted state of the instrument 10 to a manipulator, the drive input 410 receives driving forces from a drive output of the manipulator, and these driving forces drive rotation of the drive shaft 420 coupled to the drive input 410. (The driving forces may also be referred to as torques, because they induce rotational motion; herein, references to “force” are not limited to forces urging linear motion, and can include torques that urge rotational motion.) As shown in FIGS. 4 and 5, a distal end portion of each drive shaft 420 is coupled to a drive input 410, which is coupled to the chassis 311 such that the drive shaft 420 can rotate relative to the chassis 311. In addition, a proximal end portion of each drive shaft 420 may be coupled to a common proximal support 317 via a hole 318 into which a proximal end of the drive shaft 420 is inserted such that the drive shaft 420 can rotate relative to the common proximal support 317. Thus, each drive shaft 420 has one rotational bearing at a proximal end portion thereof in the form of the proximal support 317 and another rotational bearing at the distal end portion thereof in the form of the chassis 311 (via the drive inputs 410). The proximal support 317 may be coupled with the instrument shaft 100 via bearings 319 that are rotatable around axes that are perpendicular to the longitudinal axis of the shaft 100, which allows the proximal support 317 to translate along the shaft 100 along with the other portions of the force transmission assembly 300, including chassis 311. The bearings 319 also provide lateral support to the proximal support 317 and help to hold the proximal support 317 in its intended position. Additional supports (not illustrated) may also be used to couple the proximal support 317 to the chassis 311.

In addition to the drive input 410 and the drive shaft 420, each drive member 400 also comprises one or more actuation element handling components that couple the drive member 400 to at least one corresponding actuation element 500. The actuation element handling components of each drive member 400 are coupled to the drive shaft 420 thereof such that rotation of the drive shaft 420 causes the actuation element component to actuate the corresponding actuation element(s) 500 coupled thereto. In the case of the insertion drive member 400_1, the actuation element handling component comprises a rotatable drum or capstan 415 (hereinafter drum 415). In the case of the non-insertion drive members 400_2 to 400_N, their respective actuation element handling components comprise actuation transfer mechanisms 430. These actuation element handling components and their interactions with the actuation elements 500 are described in greater detail below.

### Insertion Drive Member and Corresponding Actuation Elements

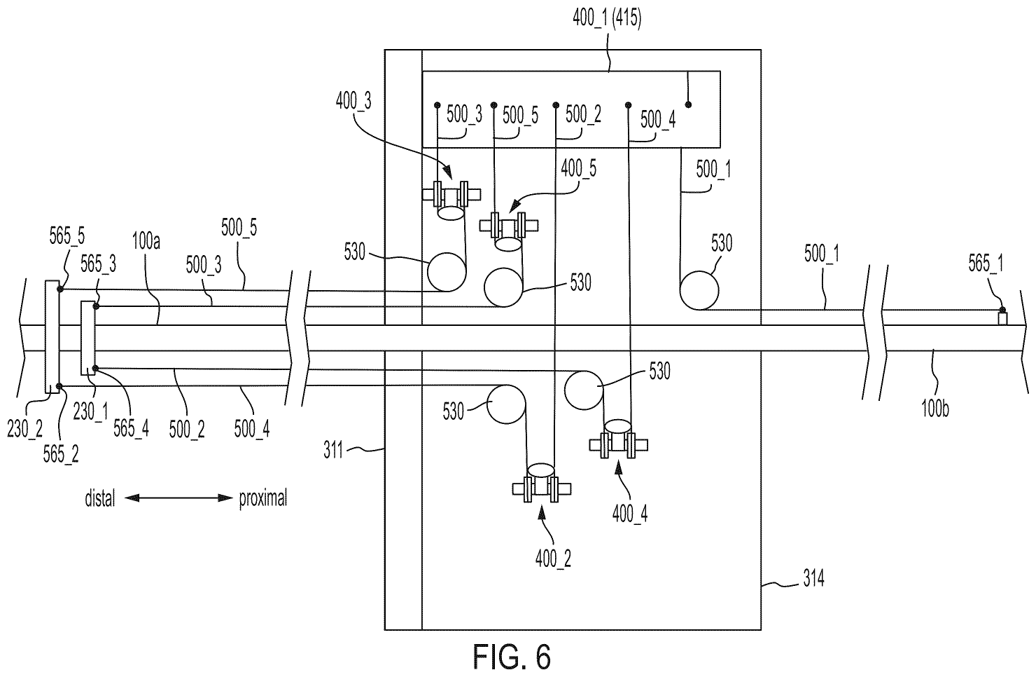

With continued reference to FIGS. 4-6, an embodiment of an insertion drive member 400_1 will be described in greater detail below. FIG. 6 is a schematic diagram showing how actuation elements 500 are routed and connected to other components of the instrument, such as the drive members 400 of the force transmission assembly 300. The diagram in FIG. 6 is not intended to show actual sizes, actual shapes, actual or relative physical locations of components, or the actual paths taken by actuation elements 500, but instead FIG. 6 shows the connections and routing schematically and in simplified form to aid understanding. It should be understood that additional handling components besides those illustrated, such as pulleys, could be interposed in the actuation element paths, and in actual implementations the actuation element paths may deviate from those shown. Moreover, as noted above, portions of the actuation elements 500 may be routed inside the instrument shaft 100, for example when passing through a distal portion 100a of the shaft 100, but in FIG. 6 the cables 500 are shown outside the shaft 100 to make them visible and to simplify the diagram.

As noted above, the insertion drive member 400_1 comprises a rotatable drum 415 to actuate (i.e., draw in and pay out) the actuation elements 500 that are coupled to the drum 415. The term “drum” as used herein is intended to refer broadly to any component having a rotational axis and a bearing surface extending generally circumferentially (although not necessarily in a perfect circle) around the rotational axis around which actuation elements 500 can be wound by rotation of the drum. The drum 415 may also be referred to as, for example, a cylinder, a spool, a capstan, a windlass, a winch, or the like. The drum 415 may have guide elements, such as grooves and/or ridges, to guide actuation elements 500 as they wind around the drum 415, or the drum 415 may omit such guide elements and have a generally smooth surface.

Multiple pullable type actuation elements 500 are coupled to and wound around drum 415 such that rotation of the drum 415 draws in or pays out the actuation elements 500, depending on direction of rotation. As shown in FIGS. 4-6, one or more of the actuation elements 500 that are coupled to the drum 415 are also coupled to and actuatable by a non-insertion drive member 400. But, as will be described in more detail below, these actuation elements 500 can move past or through the non-insertion drive members 400 to which they are coupled when the actuation elements 500 are actuated by the drum 415; therefore, the non-insertion drive members 400 can be ignored when considering the operation of the insertion drive member 400_1.

At least one of the actuation elements 500 coupled to the drum 415 (e.g., actuation element 500_1) extends proximally from the transmission assembly 300 and is coupled (directly or indirectly) to a proximal portion 100b of the shaft 100 at a position proximal of the force transmission assembly 300, as shown schematically in FIG. 6. At least one other actuation element 500 coupled to the drum 415 (e.g., actuation elements 500_2 to 500_5) extends distally from the force transmission assembly 300 and is coupled (directly or indirectly) to a distal portion 100a of the shaft 100 at a position distal of the force transmission assembly 300, as shown schematically in FIG. 6. As shown in FIGS. 4-6, the proximally extending actuation element(s) 500 are wound around the drum 415 in an opposite direction than the distally extending actuation element(s) 500. Therefore, rotation of the drum 415 in one direction draws in the proximally extending actuation element (s) 500 while simultaneously paying out the distally extending actuation element(s) 500, and rotation of the drum 415 in an opposite direction draws in the distally extending actuation element(s) 500 while simultaneously paying out the proximally extending actuation element(s) 500. Because the proximally extending actuation element(s) 500 are coupled to the proximal portion 100b of the shaft 100 at a position proximal of the transmission assembly 300, the drawing in of the proximally extending actuation element(s) 500 results in the proximally extending actuation element(s) 500 pulling shaft 100 to move in a distal direction relative to the transmission assembly 300. Conversely, because the distally extending actuation element(s) 500 are coupled to the distal portion 100a of the shaft 100 at a position distal of the transmission assembly 300, the drawing in of the distally extending actuation element(s) 500 results in the distally extending actuation element(s) 500 pulling the shaft 100 to move in a proximal direction relative to the transmission assembly 300. Thus, the insertion drive member 400_1 is actuatable to control the insertion degree of freedom of motion of the instrument 10, or in other words to drive relative motion of the shaft 100 and the force transmission assembly 300.

In the embodiment illustrated in FIGS. 4-8, the one or more proximally extending actuation element(s) 500 that are coupled to the drum 415 comprise the actuation element 500_1. The actuation element 500_1 is used to drive motion of the shaft 100 along the insertion axis in the distal direction, and thus may also be referred to herein as a distal insertion actuation element 500_1. In the embodiment of FIGS. 4-8, the distal insertion actuation element 500_1 is not coupled to any of the non-insertion drive members 400. As shown in FIGS. 5 and 6, the distal insertion actuation element 500_1 is routed from the drum 415 to a waterfall pulley 530 that has an axis of rotation perpendicular to the axis of rotation of the drum 415. The waterfall pulley 530 redirects the insertion actuation element 500_1 to extend proximally along the shaft 100. The distal insertion actuation element 500_1 extends along the shaft 100 through a groove 111 in an outer surface of the shaft 100, as shown in FIG. 5. As shown schematically in FIG. 6, the distal insertion actuation element 500_1 extends proximally until it reaches a coupling point 565_1 at which the distal insertion actuation element 500_1 is coupled either directly or indirectly to the shaft 100. The coupling point 565_1 may be part of the shaft 100 or part of some other component that is itself coupled (directly or indirectly) to the shaft 100. The coupling point 565_1 may be located somewhere along the proximal portion 100b of the shaft 100, for example at a position that remains proximal of the transmission assembly 300 throughout a range of motion of the shaft 100 and transmission assembly 300 relative to one another. Thus, when the distal insertion actuation element 500_1 is drawn in, the distal insertion actuation element 500_1 pulls on the shaft 100 and urges the shaft 100 to move in the distal direction.

In the embodiment illustrated in FIGS. 4-8, the distally extending actuation elements coupled to the drum 415 comprise actuation elements 500_2 to 500_5. In the embodiment of FIGS. 4-8, all of the distally extending actuation elements 500_2 to 500_5 coupled to the drum 415 are also coupled to another respectively corresponding drive member 400_2 to 400_5. Specifically, the cables 500_2 to 500_5 have respective proximal end portions coupled to the drum 415 and respective distal end portions coupled to coupling points 565_2 to 565_5. The actuation elements 500_2 to 500_5 extend from the drum 415 to corresponding drive members 400_2 to 4005, from the drive members 400_2 to 400_5 to corresponding waterfall pulleys 530, and then from the waterfall pulleys 530 distally along the shaft 100 to the coupling points 565_2 to 565_5. In the embodiment illustrated in FIG. 6, a first pair of actuation elements 500_2 and 500_3 are coupled to coupling points 565_2 and 565_3 that are part of a first articulable structure 230_1, and a second pair of actuation elements 500_4 and 500_5 are coupled to coupling points 565_4 and 565_5 that are part of a second articulable structure 230_2. In an embodiment, the first and second articulable structures 230 may be joints coupled in series to form a wrist mechanism that provides pitch and yaw orientations of the end effector relative to the instrument shaft 100. In other embodiments, some or all of the coupling points 565_2 to 565_5 may be parts of movable component 200 other than an articulable structure 230 (e.g., jaw members of a jaw mechanism, a knife of a vessel sealer/cutter, etc.), parts of the shaft 100, or parts of some other component of the instrument 10 which is coupled to the shaft 100, such as any moveable components that may utilize a coordinated pair of pullable actuation elements to move the moveable component.

In the embodiment of FIG. 6, the coupling points 565_2 and 565_3 of the first articulable structure 230_1 may be configured such that simultaneous actuation of the actuation elements 500_2 and 500_3 in opposite directions (e.g., by the drive members 400_2 and 400_3) drives movement of the first articulable structure 230_1 along a degree of freedom of motion (e.g., yaw). References herein to actuating two actuation elements in opposite directions mean actuating the two actuation elements such that one is drawn in while the other is simultaneously paid out, or vice versa. However, because the actuation elements 500_2 and 500_3 are coupled to the first articulable structure 230_1 so as to each individually articulate the first articulable structure 230_1 in opposite directions, if the actuation elements 500_2 and 500_3 are both drawn in simultaneously, this generates tension in both actuation elements 500_2 and 500_3 and this tension holds the first articulable structure 230_1 stationary along its degree of freedom of motion. Similarly, the coupling points 565_4 and 565_5 of the second articulable structure 2302 may be configured such that simultaneous actuation of the actuation elements 500_4 and 500_5 in opposite directions (e.g., by drive members 400_4 and 400_5) drives movement of the second articulable structure 230_2 along a degree of freedom of motion (e.g., pitch), but if the actuation elements 500_4 and 500_5 are both drawn in simultaneously then this causes the second articulable structure 230_2 to be held stationary along its degree of freedom of motion. In addition, the actuation elements 500_2 to 500_5 are all coupled to the drum 415 such that rotation of the drum 415 in one direction draws in all of the actuation elements 500_2 to 500_5 simultaneously. As noted above, when the actuation elements 500_2 to 500_5 are drawn in simultaneously, this holds the articulable structures 230_1 and 230_2 stationary along their degrees of freedom of motion, and therefore the drawing in of the cables 500_2 to 500_5 generates a net force that urges the articulable structures 230_1 and 2302, and therefore the shaft 100 to which they are coupled, to translate together proximally along the insertion axis relative to the transmission assembly 300. The actuation elements 500_2 to 500_5 can thus also be referred to as proximal insertion actuation elements 500.

In some embodiments, the transmission assembly 300 may include another actuation element (not illustrated), which is dedicated for driving the proximal translation of the shaft 100 along the insertion axis and not for driving any other movable components 200. For example, such an actuation element may be coupled to the drum 415 and extend distally from the force transmission assembly 300 to couple directly to a distal portion 100a of the shaft 100, such that pulling in the actuation element 500 causes proximal translation of the shaft 100.

### Non-Insertion Drive Members and Corresponding Actuation Elements

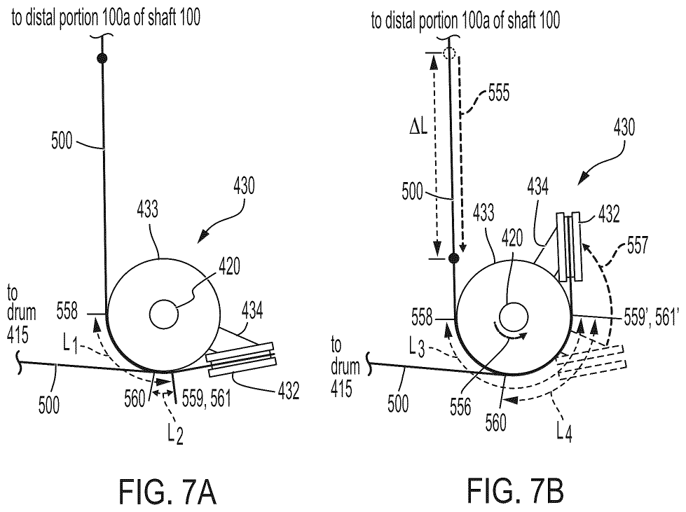

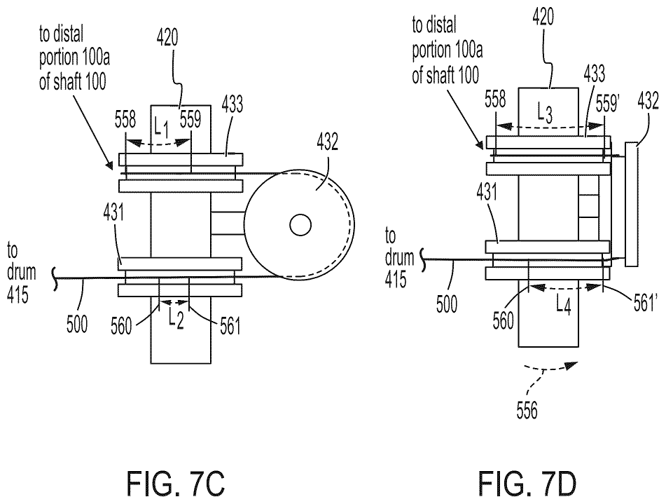

With reference to FIGS. 4, 5, 7A, and 7B the non-insertion drive members 400_2 to 400_N will be described in greater detail below. FIGS. 7A and 7B illustrate plan views of an actuation transfer mechanism 430 (described in greater detail below) in two states from a perspective above (i.e., proximal of) the actuation transfer mechanism 430.

As noted above, at least some of the non-insertion drive members 400_2 to 400_N comprise actuation transfer mechanisms 430 to couple with and actuate actuation elements 500_2 to 500_N, as shown in FIGS. 4, 5, and 7. In FIGS. 4-6, all of the insertion drive members 400_2 to 400_5 comprise an actuation transfer mechanism 430, but in other embodiments some drive members 400 might not have an actuation transfer mechanism 430. In addition, some drive members 400 may have more than one actuation transfer mechanism 430, enabling them to actuate more than one actuation element 500 at a time.

An actuation transfer mechanism 430 of a given drive member 400 is configured to allow both the insertion drive member 400_1 and the given drive member 400 to actuate the same actuation element 500 independently of one another. In other words, if the drum 415 is held stationary while a given drive member 400 is rotated, the actuation transfer mechanism 430 of the given drive member 400 actuates (pays out or draws in) the corresponding actuation element 500. Likewise, if the drum 415 is rotated while the given drive member 400 is held stationary, the actuation transfer mechanism 430 allows the drum 415 to actuate the actuation element 500. Thus, the actuation transfer mechanism 430 can allow a transfer of actuation of the same actuation element 500 between the drum 415 and the corresponding drive member 400 to which the actuation element 500 is coupled.

In the description below, the actuation transfer mechanism 430 of the drive member 400_3 is described as an example, but it should be understood that the description is also applicable to the actuation transfer mechanisms 430 of the other non-insertion drive members 400_2 to 400_N and their corresponding actuation elements 500_2 to 500_N. As shown in FIGS. 4 and 5, in some embodiments the actuation transfer mechanism 430 comprises a first pulley 431 coupled to and coaxial with the drive shaft 420, a second pulley 432 radially offset from the drive shaft 420 and having an axis of rotation oriented transverse to an axis of rotation of the drive shaft 420, and a third pulley 433 coupled to and coaxial with the drive shaft 420. The first and third pulleys 431 and 433 are rotatable relative to the drive shaft 420 (e.g., the pulleys 431 and 433 may comprise ball bearings or other bearings that permit relative rotation). The second pulley 432 is attached to an arm member 434 coupled to and extending generally radially from the drive shaft 420 and constrained to rotate with the drive shaft 420. The actuation element 500_3 is routed from the drum 415 to the first pulley 431 such that the actuation element 500_3 begins to wrap around the drive shaft 420 in a first direction, then the actuation element 500_3 is looped around the second pulley 432 and redirected back to the third pulley 433 to wrap around the drive shaft 420 again in a second direction opposite the first direction. From the third pulley 433, the actuation element 500_3 extends to a waterfall pulley 530 which redirects the actuation element 500_3 to extend along the shaft 100.

The actuation transfer mechanism 430 as described above allows the actuation element 500_3 to move past or through the non-insertion drive member 400_3 when actuated by the insertion drive member 4001. In particular, if the actuation element 500_3 is actuated by the insertion drive member 400_1, the pulleys 431, 432, and 433 rotate around their respective axes in response to actuation of the actuation element 500_3, thereby allowing the actuation element 500_3 to be paid out or drawn in through the actuation transfer mechanism 430 without actuation (rotation) of the drive member 400_3. Thus, though the drive member 400_3 is held stationary, the actuation element 500_3 can nevertheless be actuated, for example by the insertion drive member 400_1. In other words, the actuation transfer mechanism 430 allows the insertion drive member 400_1 to actuate the actuation element 500_3 independently of the drive member 400_3. The same is true of the other drive members 400_2 to 400_5, and therefore the insertion drive member 400_1 can actuate all of the actuation elements 500_2 to 500_5 together to drive the insertion degree of freedom of motion independently of actuation of the drive members 400_2 to 400_5.

On the other hand, the actuation transfer mechanism 430 as described above also allows the drive member 400_3 to actuate the actuation element 500_3, notwithstanding the fact that the actuation element 500_3 is free to move through the actuation transfer mechanism 430. In particular, rotation of the drive shaft 420 causes the arm 434 to rotate with the shaft 420 and this causes the second pulley 432 coupled to the arm 434 to revolve around the drive shaft 420. Because the actuation element 500_3 is looped around the second pulley 432, the revolution of the second pulley 432 around the drive shaft 420 pays out or draws in the actuation element 500_3, depending on the direction of motion. Specifically, revolution of the second pulley 432 around the drive shaft 420 in a one direction (clockwise in the case of the drive member 400_3), increases the amount of the actuation element 500_3 that is wound around the first and third pulleys 431 and 433, thus drawing in the actuation element 500_3. Conversely, revolution of the second pulley 432 around the drive shaft 420 in an opposite direction (counterclockwise in the case of the drive member 400_3) decreases the amount of the actuation element 500_3 that is wound around the first and third pulleys 431 and 433, thus drawing in the actuation element 500_3. Thus, the drive member 400_3 can actuate the actuation element 500_3 even if the insertion drive member 400_1 is held stationary. In other words, the actuation transfer mechanism 430 allows the drive member 400_3 to actuate the actuation element 500_3 independently of the insertion drive member 400_1.

In the illustrated embodiment, the actuation elements 500_2 and 500_3 are coupled to the same movable component 200 (e.g., articulable structure 230_1) to drive motion in opposite directions along a degree of freedom of motion. Thus, in such embodiments the drive member 400_2 may be actuated in coordination with the actuation of drive member 400_3, such that the actuation elements 500_2 and 500_3 are actuated in a coordinated fashion (e.g., one is drawn in while the other is drawn out) to actuate the articulation degree of freedom motion of the articulable structure 230_1.

The manner in which a drive member 400 actuates an actuation element 500 via the actuation transfer mechanism 430 can be better understood by considering FIGS. 7A-7D, which illustrate schematically and in greater detail an actuation transfer mechanism 430. FIGS. 7A and 7B illustrate the actuation transfer mechanism 430 in a plan view from a perspective proximal of the actuation transfer mechanism 430, while FIGS. 7C and 7D illustrate a side view of the actuation element 430. In FIGS. 7A and 7C, the actuation transfer mechanism 430 is in a first state, and in FIGS. 7B and 7D the actuation transfer mechanism 430 is in a second state, in which the drive shaft 420 has been rotated counterclockwise (in the view of the figure) relative to the first state, as indicated by the arrow 556. The rotation of the drive shaft 420 causes the second pulley 432 to revolve counterclockwise around the drive shaft 420, as indicated by the arrow 557 in FIGS. 7B and 7D. In the first state shown in FIGS. 7A and 7C, the portion of the actuation element 500 that is wound around the third pulley 433 has a length L₁ from where it initially meets the third pulley 433 (location 558) to where it leaves the third pulley 433 (location 559) to extend to the second pulley 432, and the portion of the actuation element 500 that is wound around the first pulley 431 has a length L₂ from where it initially meets the first pulley 431 (location 560) to where it leaves the first pulley 431 to extend to the second pulley 432 (location 561). In the second state shown in FIGS. 7B and 7D (after rotation of the drive shaft 420), the portion of the actuation element 500 that is wound around the third pulley 433 has a length L₃ from where it initially meets the third pulley 433 (location 558) to where it leaves the third pulley 433 (location 559′) and the portion of the actuation element 500 that is wound around the first pulley 431 has a length L₄ from where it initially meets the first pulley 431 (location 560) to where it leaves the first pulley 431 (location 561′). As can be seen by comparing the lengths L₁ and L₂ illustrated in FIG. 7A with the lengths L₃ and L₄ illustrated in FIG. 7B, the rotation of the drive member 400 about the drive shaft 420 increases the total length of the actuation element 500 that is wound around the first and third pulleys 431 and 433. Specifically, the total length of the actuation element 500 that is wound around the first and third pulleys 431 and 433 in the first state is L₁+L₂, while in the second state it is L₃+L₄, where L₄>L₂ and L₃>L₁. Thus, the total length of the actuation element 500 that is wound around the first and third pulleys 431 and 433 has increased by an amount ΔL=L₃+L₄−(L₁+L₂) between the first and second states. Because one end portion of the actuation element 500 is attached to the drum 415 and thus held stationary, the other end portion of the actuation element 500, which is attached to a distal portion 100a of the shaft 100 (e.g., via a movable component 200), will be drawn in by the distance ΔL to provide for the increased length of the actuation element 500 wrapped round the first and third pulleys 431 and 433. The opposite would occur if the drive shaft 420 were rotated in the opposite direction (clockwise in the view of FIGS. 7A-7B), i.e., the length of the actuation element 500 wound around the pulleys 431 and 433 would shorten, resulting in the actuation element 500 paying out.

In the illustrated embodiment, the pulleys 431, 432, and 433 are rotatable around their axes of rotation, which may reduce friction and makes actuation of the actuation elements 500 easier. However, in some embodiments one, some, or all of the pulleys 431, 432, and 433 could be replaced with non-rotating bearings. Operation of the actuation transfer mechanism 430 would be the same in such an embodiment, except that the actuation element 500 would slide relative to the bearings rather than the pulleys 431, 432, and 433 rotating. This may result in increased friction, but in some circumstances this may be acceptable.

The embodiment of FIG. 3-7D is described above as one non-limiting example of how the drive members 400 may be arranged to interact with the actuation elements 500, but other embodiments are contemplated herein. Specifically, although five drive members 400 are illustrated in FIGS. 3-8, other embodiments have more or fewer drive members 400, including any number of drive members 400_1 to 400_N where N is any integer equal to or greater than 2. In addition, although FIGS. 3-8 illustrates five actuation elements 500 with one extending proximally and four extending distally, in other embodiments more or fewer actuation elements 500 may be provided that extend in the proximal direction and more or fewer actuation elements 500 may be provided that extend in the distal direction. Furthermore, although FIGS. 3-8 illustrate all of the non-insertion drive members 400 as being coupled to distally extending actuation elements 500, in other embodiments one or more non-insertion drive members 400 may be coupled to one or more of the proximally extending actuation elements 500. Moreover, although FIGS. 3-8 illustrate all of the actuation elements 500 that are driven by the non-insertion drive members 400_1 to 400_5 as also being coupled to the insertion drive member 400_1, in other embodiments one or more of the actuation elements 500 may be coupled to a non-insertion drive member 400 without also being coupled to the insertion drive member 400_1. In addition, although FIG. 6 illustrates all of the drive actuation elements 500 that extend distally as being coupled to an articulable structure 230 in the form of a wrist supporting an end effector, in other embodiments one or more actuation elements 500 that extend distally may be coupled to something other than such an articulable structure 230, such as another type of movable component 200 (e.g., a movable component of an end effector 210), an articulable structure located along a lengthy of the shaft 100 to provide a degree of freedom of articulation of the shaft, another actuation element (e.g., push-pull rod), or another component of the instrument 10. Furthermore, although FIG. 3-7 illustrate all of the actuation elements 500 that extend distally as being coupled to a non-insertion drive member 400, in some embodiments one or more actuation elements 500 that extend distally are coupled to the insertion drive member 400_1 without being coupled to another non-insertion drive member 400.

### Bearing Mechanisms for Routing Actuation Elements

With continued reference to FIGS. 4 and 5, an embodiment of a bearing mechanism 440, such as those mentioned above, is described below in greater detail. As described above, at least one of the non-insertion drive members 400 is provided with a bearing mechanism 440 coupled to the drive shaft 420 of the drive member 400. The bearing mechanism 440 is configured to redirect an actuation element 500 that is actuatable by another drive member 400 as that actuation element 500 extends between the insertion drive member 400_1 and the other drive member 400. Specifically, as shown in FIG. 4, the drive member 400_2 is provided with a bearing mechanism 440, which redirects the actuation element 500_3 as the actuation element 500_3 extends between the drum 415 and the drive member 400_3 to which the actuation element 500_3 is coupled for actuation. Similarly, the drive member 400_5 is provided with a bearing mechanism 400, which redirects the actuation element 500_4 as the actuation element 500_4 extends between the drum 415 and the drive member 400_4 to which the actuation element 500_4 is coupled for actuation, as shown in FIG. 4. The bearing mechanism 440 may be positioned on the drive shaft 420 distal to the actuation transfer mechanism 430, such as in the case of the drive member 400_3 in FIG. 4, or the bearing mechanism 440 may be positioned on the drive shaft 420 proximal to the actuation transfer mechanism 430, such as in the case of the drive member 400_5 in FIG. 5. The location of the bearing mechanism 440 along the drive shaft 420 may be selected to align with the actuation element 500 that is routed by the bearing mechanism 440, the location of which may be arbitrarily chosen based on the overall configuration of the drive members, actuation element paths, and other components of the force transmission assembly. The bearing mechanism 440 may have a generally cylindrical portion with an outward facing surface thereof acting as a bearing surface that engages with and redirects the actuation element 500.

In some embodiments, the bearing mechanism 440 may be coupled to the drive shaft 420 such that the bearing mechanism 440 can rotate relative to the drive shaft 420. Thus, the bearing mechanism 440 may act as a pulley, rotating as the actuation element 500 engaged therewith moves past the drive member 400, such as when the actuation element 500 is actuated by the drum 415. The bearing mechanism 440 may comprise an internal bearing (not shown) that rotatably couples the bearing mechanism 440 to the drive shaft 420. In some embodiments, the internal bearing may comprise a low friction rotatable bearing, such as a ball bearing, deep-groove ball bearings, angular contact bearings, cylindrical roller bearings, thrust bearings, tapered roller bearings, etc., to reduce the amount of friction resisting rotation of the bearing mechanism 440. In other embodiments, the internal bearing may comprise a plain bearing. In some embodiments, grease, oil, or other lubricants may be used with the internal bearing reduce friction and wear. Material combinations of the internal bearing and the drive shaft 420, or surface treatments applied thereon, may also be chosen to reduce friction, wear, or galling. For example, relative low friction (lubricious) materials such as PTFE, UHMWPE, Acetal, brass or bronze, Nitronic 60, Nyon, stainless steel, or other similar materials may be used for a body of the bearing mechanism 440 and/or the drive shaft 420 to reduce the amount of friction resisting rotation of the bearing mechanism 440.

Because the bearing mechanisms 440 are provided on the drive members 400_2 and 4005, the drive members 400_2 and 400_5 can be positioned at locations of the force transmission assembly 300 that might otherwise not be feasible. For example, as shown in FIG. 4, the drive member 4002 is positioned at a location between the drive member 400_3 and the drive member 400_1 at which the drive member 400_2 would have otherwise interfered with the actuation element 500_3 as it extended between the drum 415 and the drive member 4003, but because the bearing mechanism 440 is provided on the drive member 400_2 the actuation element 500_3 is redirected around the drive member 4002. Similarly, the drive member 400_5 can be positioned between the drive member 400_4 and the drive member 400_1 due to the bearing mechanism 440, as shown in FIG. 5. This ability to position the drive members 400 relative to one another at locations that would otherwise not be feasible can allow the transmission assembly 300 to be made smaller, or allow more free space in which additional components could be included at a given size/footprint of transmission assembly 300.

### Biasing Devices for Taking Up Actuation Element Slack

With continued reference to FIGS. 4 and 5, a biasing device 450, which is an embodiment of the biasing device mentioned above, is described below in greater detail. As noted above, in some embodiments a biasing device 450 is provided for at least some of the non-insertion drive members 400 to prevent or take up slack that might otherwise develop in the actuation elements 500 driven by those drive members 400 in an unmounted state of the instrument to the manipulator. In the embodiment of FIGS. 4 and 5, a given biasing device 450 comprises two biasing elements in the form of torsion springs 451a and 451b, which are coupled, respectively, to the drive members 400 of a pair of drive members that drive motion of the same movable component 200 along opposite directions of a given degree of freedom of motion. For example, in the embodiment of FIGS. 4 and 5, one biasing device 450 may be coupled to the drive members 400_2 and 400_3, which are paired together to drive motion of a first movable component 200 (e.g., the first articulable structure 230_1) along opposite directions of the same degree of freedom of motion, and another biasing device 450 may be provided for the drive members 400_4 and 400_5, which are paired together to drive motion of a second movable component 200 (e.g., the second articulable structure 230_2) along opposite directions of the same degree of freedom of motion.

The biasing device 450 is configured to bias the drive shafts 420 of the corresponding pair of drive members 400 to which the biasing device 450 is coupled towards rotation in respective directions that draw in the respective actuation elements 500 coupled thereto. In other words, the biasing device 450 is configured to bias the drive shafts 420 of the corresponding pair of drive members 400 such that, if slack were to develop in the actuation element 500 coupled to one of the drive members 400, the biasing device 450 would cause rotation of that drive member 400 so as to take up that slack. Thus, if an external force causes a first drive member 400 of the pair to pay out an actuation element 500, the biasing device 450 urges the second drive member 400 of the pair to draw in the other actuation element 500, and vice versa.

In embodiments in which a pair of the drive members 400 rotate in opposite directions to draw in the actuation elements 500 coupled thereto (such as in FIGS. 4 and 5), the biasing device 450 may be configured to bias the drive members 400 in opposite directions relative to one another. In contrast, in embodiments in which the pair of the drive members 400 rotate in the same direction to draw in the actuation elements 500 coupled thereto (not illustrated), the biasing device 450 may be configured to bias the drive members 400 in the same direction relative to one another.

The biasing device 450 coupled to the drive members 400_2 and 400_3 as shown in FIG. 4 will be described below as an illustrative example. As shown in FIG. 4, in some embodiments the biasing device 450 comprises a first torsion spring 451a coupled to one drive member 400 of the pair (e.g., drive member 400_2), and a second torsion spring 451b coupled to the other drive member 400 of the pair (e.g., drive member 400_3). More specifically, the first and second torsion springs 451a and 451b are coupled (directly or indirectly) to the drive shaft 420 of one of the drive members 400. For example, in FIGS. 4 and 5, the first and second torsion springs 451a and 451b are coupled to the drive inputs 410 of the drive members 400_2 and 400_3. In other embodiments, the first and second torsion springs 451a and 451b may be coupled to other parts of the drive members 400, such as directly to the drive shafts 420. The opposite ends of the first and second torsion springs 451a and 451b may be held stationary. For example, in the embodiment of FIGS. 4 and 5, the first and second torsion springs 451a and 451b are coupled together by a linkage 452, which holds the ends of the springs 451a and 451b stationary. In other embodiments (not illustrated), the linkage 452 is omitted and the first and second torsion springs 451a and 451b are anchored to the chassis 311.

The first and second torsion springs 451a and 451b are each configured to urge rotation of one of the drive members 400 in a direction that draws in the actuation elements 500 coupled to the drive member 400. In other words, the first and second torsion springs 451a and 451b are configured to bias the drive members 400 so as to take up any slack which might otherwise develop in the actuation elements 500 coupled thereto. For example, if slack were to begin developing in the actuation element 500_2 (due, for example, to an external force being applied to movable component 200), the first torsion spring 451a would urge the drive member 400_2 to rotate clockwise (from the perspective of FIG. 4) so as to take up the slack. On the other hand, if slack were to begin developing in the actuation element 500_3 (due, for example, to a different external force being applied to movable component 200), the second torsion spring 451b would urge the drive member 400_4 to rotate counterclockwise (from the perspective of FIG. 4) so as to take up the slack.

In the embodiment illustrated in FIGS. 4 and 5, the drive members 400_2 and 400_3 have a reversed configuration relative to one another, meaning that the drive members 400_2 and 400_3 are configured such that they are rotatable in opposite directions as one another to pay out their respective actuation elements 500_2 and 500_3 and they are rotatable in opposite directions as one another to draw in their respective actuation elements 500_2 and 500_3. For example, to draw in the actuation element 500_2, the drive member 400_2 is rotated in a clockwise direction, while the drive member 400_3 is rotated in a counterclockwise direction to draw in the actuation element 500_3. This configuration of the drive members 400_2 and 400_3 in which they have reversed actuation directions is achieved because their respective actuation transfer mechanisms 430 have a reversed orientation relative to one another such that the actuation elements 500_2 and 500_3 are wrapped in opposite directions around the drive members 400_2 and 400_3, respectively. Thus, because the drive members 400_2 and 400_3 have reversed actuation directions relative to one another in this embodiment, the biasing device 450 is configured to bias the pair of drive members 400_2 and 4003 in opposite directions, as noted above. If instead the drive members 400_2 and 400_3 were rotatable in the same direction to draw in their actuation elements 500_2 and 5003, the biasing device 450 is configured to bias the pair of drive members 400_2 and 400_3 in the same direction.

As described above, the biasing devices 450 can prevent or take up slack in the actuation elements 500 in an unmounted state of the instrument 10. The biasing devices 450 may reduce or prevent (take up) slack that would otherwise occur in a given actuation element 500 when a movable component 200 is moved because the biasing device 450 forces the drive member 400 coupled to that given actuation element 500 that would otherwise have developed slack to rotate so as to draw in the given actuation element 500, thus taking up the slack.

### Sealing Structures

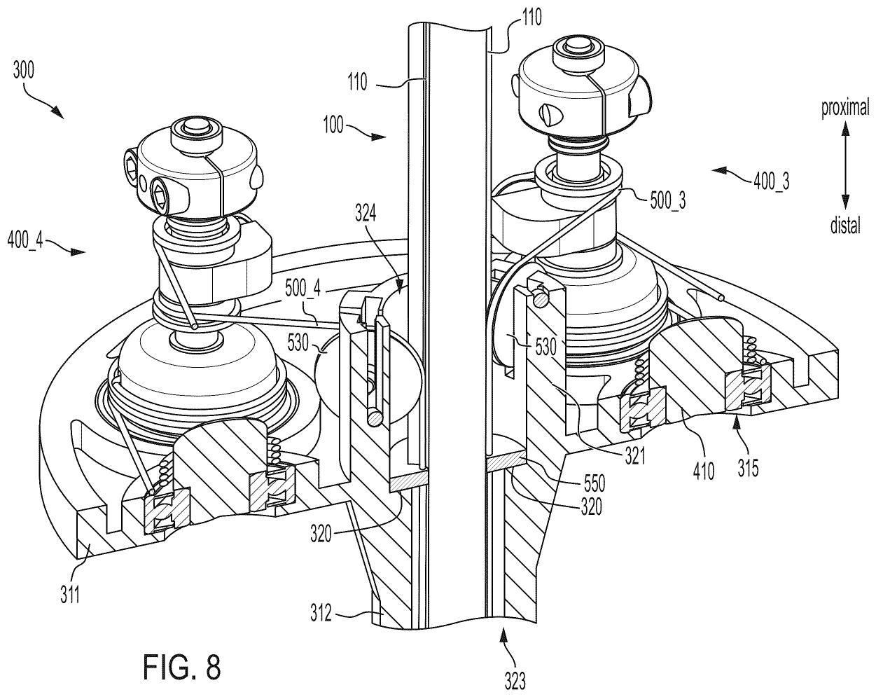

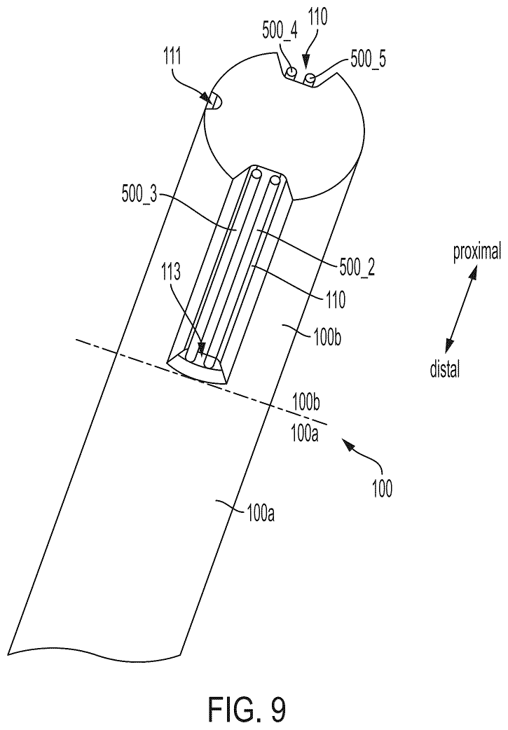

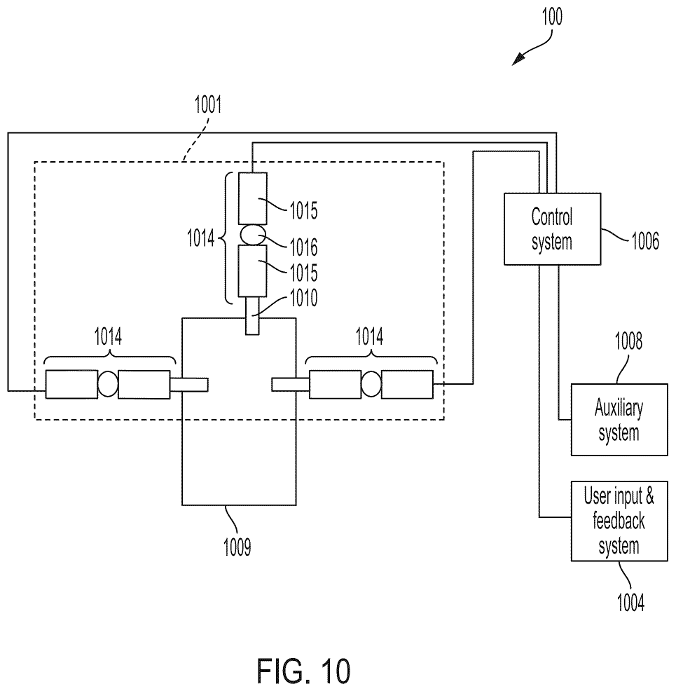

With reference to FIGS. 8 and 9, a seal 550, which may be used as the seal 34 mentioned above, is described below in greater detail. FIG. 8 shows a partial perspective, partial cross-section of the instrument 10 taken along line 7 in FIG. 5. FIG. 9 shows a perspective view of a portion of the shaft 100 of the instrument 10.

As described above, in some embodiments the seal 550 is provided to prevent escape of fluid, such as insufflation gasses, for example, from a workspace through the instrument 10. As shown in FIG. 8, the seal 550 is provided in a bore 323 of the chassis 311 and sleeve 312 of the force transmission assembly 300 through which the shaft 100 and proximally extending cables 500 extend. The seal 550 is positioned between an interior surface of the bore 323 and an exterior surface of the shaft 100. More specifically, the seal 550 may be positioned at a proximal end of the sleeve 312 of the housing 310 near or at a position where the sleeve 312 meets the chassis 311.

The seal 550 may be generally annular in shape, but an inner surface thereof may be irregular instead of circular such that the inner surface conforms to contours of the exterior surface of the shaft 100, including extending into and conforming to actuation element routing grooves 110 in the shaft 100, described in greater detail below. The outer surface of the seal 550 contacts interior surfaces of the housing 310 within the bore 323. Thus, the seal 550 creates a seal within the bore 323 between the housing 310 and the shaft 100.

As shown in FIGS. 2 and 9, the instrument shaft 100 may comprise a distal portion 100a and a proximal portion 100b. In some embodiments, distally extending actuation elements 500 are routed within the interior of the shaft 100 in the distal portion 100a and proximally extending actuation elements 500 are also routed along an exterior of the shaft along the proximal portion 100b. As shown in FIG. 9, the shaft 100 may comprise one or more actuation element routing features along the exterior surface of the proximal portion 100b of the shaft 100. These actuation element routing features can include longitudinal grooves 110 and 111 in the outer surface of the proximal portion 100b of the shaft 100. The grooves 110 and 111 are configured to receive and route the actuation elements 500 along the exterior surface of the shaft 100. Specifically, the grooves 110 are configured to receive and route distally extending actuation elements 500 (e.g., actuation elements 500_2 to 500_5), while the groove 111 is configured to receive and route a proximally extending actuation element (e.g., actuation element 500_1). The grooves 110 terminate at transition point between the distal portion 100a and the proximal portion 100b of the shaft 100. The grooves 110 terminate in openings 113, which communicate with an interior cavity in the shaft 100. Thus, the distally extending actuation elements 500 routed along the exterior surface of the shaft 100 in the grooves 110 may transition to being routed within the interior of the shaft 100 via the openings 113. The groove 111 may terminate at any desired location along the shaft 100, depending on the desired range of motion of the shaft 100 relative to the transmission assembly 300. The groove 111 does not terminate at an opening, as the proximally extending actuation elements 500 do not pass into the interior of the shaft 100 in the illustrated embodiment. Although the grooves 110 and 111 are illustrated in particular locations and shapes in FIG. 9, it should be understood that the grooves 110 and 111 could be located in other positions (e.g., based on a desired routing of the actuation elements 500), could have different shapes or sizes (e.g., based on the configuration of the actuation elements 500). Moreover, more or fewer grooves could be included, depending on actuation elements arrangements. In some embodiments, the grooves 110 and/or 111 may be omitted.

As shown in FIG. 8, in some embodiments the bore 323 may comprise a ledge 320. The ledge 320 may be positioned near or at a location where the sleeve 312 meets the chassis 311. A ring wall 321 may extend proximally from the ledge 320 into the chamber defined by the chassis 311 and the cover portion 314. The ring wall 321 defines a proximal portion of the bore 323. Waterfall pulleys 530 may be coupled to the ring wall 321, with actuation elements 500 extending through openings into the ring wall 321 to enter the bore 323. The seal 550 may be disposed on a proximal facing surface of the ledge 320.

As noted above, the sleeve 312 of the housing 310 is configured to be insertable into a passage of a manipulator when the instrument 10 is mounted to the manipulator. The sleeve 312 facilities sealing of the workspace to prevent escape of fluid, such as insufflation gasses or other fluids. In particular, a second seal (not illustrated) may be positioned between the exterior surface of the sleeve 312 and an interior surface of the passage of the manipulator or a cannula coupled to the manipulator. Thus, the seal 550 described above seals the interior side of the sleeve 312 relative to the shaft 100 and the second insufflation seal seals an exterior side of the sleeve 312 relative to the manipulator or cannula. Accordingly, the seal 550 and the second insufflation seal together are able to prevent escape of fluid from a worksite (e.g., a body cavity) around the instrument to an environment external to the worksite. In some examples, additional seals (not illustrated) may exist at other openings, such as at the movable component 200 (e.g., at a wrist thereof), to likewise prevent ingress or escape of fluid.

In some embodiments, the instrument 10 may be configured such that the cable opening 113 described above remains within the sleeve 312, more specifically between the seal 550 and a distal end portion of the sleeve 312, throughout a full range of motion of the instrument 10 along the instrument shaft 100. This may ensure that a proper seal is maintained throughout regardless of where the shaft 100 is located along its range of motion.

### Manipulator System

FIG. 10 is a schematic block diagram of an embodiment of a computer-assisted instrument control system 1000 for remote control of instruments in accordance with various embodiments, also referred to herein as a teleoperable instrument system. Such a system can be a medical system that employs robotic technology, as those having ordinary skill in the art are familiar with. The system 1000 comprises a manipulator assembly 1001, a control system 1006, and a user input and feedback system 1004. The system 1000 may also include an auxiliary system 1008 to provide various supporting functionality to the instruments or the overall system. These components of the system 1000 are described in greater detail blow.

The manipulator assembly 1001 comprises one or more manipulators 1014. FIG. 10 illustrates three manipulators 1014, but any number of manipulators 1014 may be included. While a manipulator may comprise a single mechanical link, in the embodiment of FIG. 10, each manipulator 1014 comprises a kinematic structure of two or more links 1015 coupled together by one or more joints 1016. The joints 1016 may impart various degrees of freedom of movement to the manipulator 1014, allowing the manipulator 1014 to be moved around a workspace 1009. For example, some joints 1016 may provide for rotation of links 1015 relative to one another, other joints 1016 may provide for translation of links 1015 relative to one another, and some may provide for both rotation and translation. Some or all of the joints 1016 may be powered joints, meaning a powered drive element may control movement of the joint 1016 through the supply of motive power. Such powered drive elements may comprise, for example, electric motors, pneumatic or hydraulic actuators, etc. Additional joints 1016 may be unpowered joints. FIG. 2 illustrates each manipulator 1014 as having two links 1015 and one joint 1016, but in practice a manipulator may include more links 1015 and more joints 1016, depending on the needs of the system 1000. The more links 1015 and joints 1016 are included, the greater the degrees of freedom of movement of the manipulator 1014.

Each manipulator 1014 may be configured to support and operate one or more instruments 1010. The instruments 1010 may include various types of instruments, including for example industrial instruments and medical instruments (e.g., surgical instruments, imaging instruments, diagnostic instruments, therapeutic instruments, etc.). For example, the instruments 1 and 10 described above may be used as any of the instruments 1010. A manipulator 1014 may comprise an instrument manipulator interface to which an instrument 1010 can be removably coupled. The instrument manipulator interface may be located, for example, at a distal end portion of the manipulator 1014. The instrument manipulator interface may include drive outputs to provide driving forces to drive inputs of the instrument 1010 to control operations of the instrument 1010, such as moving an end-effector of the instrument, opening/closing jaws, driving translating and/or rotating components, etc. The drive outputs may be driven by actuators (e.g., electrical motors, hydraulic actuators, pneumatic actuators, etc.) and may interface with and mechanically transfer driving forces to corresponding drive inputs of the instrument 1010 (directly, or via intermediate drive outputs, which may be part of a sterile instrument adaptor (ISA) (not illustrated)). The ISA may be placed between the instrument 1010 and the instrument interface to maintain sterile separation between the instrument 1010 and the manipulator 1014. The instrument interface may also comprise other interface components (not illustrated), such as electrical interfaces to provide and/or receive electrical signals to/from the instrument 1010. In some embodiments, the manipulator assembly can include flux delivery transmission capability as well, such as, for example, to supply electricity, fluid, vacuum pressure, light, electromagnetic radiation, etc. to the end effector. In other embodiments, such flux delivery transmission may be provided to an instrument through another auxiliary system, described further below.

An example embodiment of an instrument manipulating portion 60 of a manipulator 1014, is illustrated in FIG. 10. The instrument manipulating portion 60 is configured to support and operate an instrument 10 mounted thereon. As shown in FIG. 11, the instrument manipulating portion 60 comprises a base 77 and an instrument holder assembly 70. The base is coupled with a link 1015 of a manipulator 1014, for example by a joint 1016. The instrument holder assembly 70 comprises an outer housing 72 coupled to the base 77 and an instrument holder 71 coupled to the outer housing 72. The instrument holder 71 comprises an interface 73 and an inner portion (not illustrated) that is received within the outer housing 72. The instrument holder 71 may be rotatable relative to the outer housing 72, thus imparting a roll degree of freedom of motion to an instrument 10 mounted thereon.

As shown in FIG. 11, the instrument holder 71 comprises an interface 73 configured to receive and interface with the chassis 311 of the instrument 10 in a mounted state. The interface 73 comprises drive outputs 75 configured to interface with the drive inputs of the instrument, such as drive inputs 410 of the instrument 10, in a mounted state of the instrument to the manipulator. The drive outputs 75 may interface directly with the drive inputs 410, or the drive outputs 75 may interface indirectly with the drive inputs via intermediate drive outputs of an ISA (i.e., the drive outputs 75 engage corresponding intermediate drive outputs of the ISA, and the intermediate drive outputs of the ISA engage corresponding drive inputs 410). The drive outputs 75 may have a shape that is complementary to a shape of the drive inputs 410 and/or a shape of the intermediate drive outputs to allow for mating engagement such that rotation of the drive outputs 75 drives rotation drive inputs 410 and/or intermediate drive outputs. The inner portion of the instrument holder 71 may comprises actuators (not illustrated) coupled to the drive outputs 75 to supply torque to the drive outputs 75. Motive power, such as electricity or pressurized hydraulic or pneumatic fluid, may be provided to the actuators via power supply lines, which may be routed through the base 77 and into the housing 72.

The instrument holder assembly 70 of the embodiment of FIG. 11 comprises a passage 76 extending through the instrument interface 73 and the outer housing 72. In FIG. 11, the passage 76 comprises a generally cylindrical bore, but in other embodiments the passage 76 may have other shapes, such as a slot with a U-shaped cross-section that is open along a lateral side of the instrument holder assembly 70 in addition to being open at opposite axial ends of the instrument holder assembly 70. A portion of the shaft 100 and the sleeve 312 are received in the passage 76 in the mounted state. As described above, the sleeve 312 comprises alignment features 316a and 316b, and the inner surface of the passage 76 may comprise complementary alignment features (not illustrated) to engage with the alignment features 316a and 316b so as to progressively align the instrument 10 as the sleeve 312 is inserted farther into the passage 76.

As shown in FIG. 11, a cannula 74 may be coupled to (or may be an integral part of) the instrument manipulating portion 60. The cannula 74 may be configured for insertion into a patient through an incision or natural orifice so as to allow the shaft 100 of the instrument to be inserted and advanced therethrough. As noted above, a distal end portion of the sleeve 312 of an instrument 10 may extend partially into the cannula 74, and a seal may be provided between the cannula 74 and the exterior of the sleeve 312.

The system 1000 can also include a user input and feedback system 1004 operably coupled to the control system 1006. The user input and feedback system 1004 comprises one or more input devices to receive input control commands to control operations of the manipulator assembly 1001. Such input devices may include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device may be provided with the same degrees of freedom as the associated instrument that they control, and as the input device is actuated, the instrument, through drive inputs from the manipulator assembly, is controlled to follow or mimic the movement of the input device, which may provide the user a sense of directly controlling the instrument. Telepresence input devices may provide the operator with telepresence, meaning the perception that the input devices are integral with the instrument. The user input and feedback system 1004 may also include feedback devices, such as a display device (not shown) to display images (e.g., images of the workspace 1009 as captured by one of the instruments 1010), haptic feedback devices, audio feedback devices, other graphical user interface forms of feedback, etc.

The control system 1006 may control operations of the system 1000. In particular, the control system 1006 may send control signals (e.g., electrical signals) to the manipulator assembly 1001 to control movement of the joints 1016 and to control operations of the instruments 1010 (e.g., through drive interfaces at the manipulators 1014). In some embodiments, the control system 1006 may also control some or all operations of the user input and feedback system 1004, the auxiliary system 1008, or other parts of the system 1000. The control system 1006 may include an electronic controller to control and/or assist a user in controlling operations of the manipulator assembly 1001. The electronic controller comprises processing circuitry configured with logic for performing the various operations. The logic of the processing circuitry may comprise dedicated hardware to perform various operations, software (machine readable and/or processor executable instructions) to perform various operations, or any combination thereof. In examples in which the logic comprises software, the processing circuitry may include a processor to execute the software instructions and a memory device that stores the software. The processor may comprise one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In examples in which the processing circuitry includes dedicated hardware, in addition to or in lieu of the processor, the dedicated hardware may include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry may also include any combination of dedicated hardware and processor plus software.

As noted above, differing degrees of user control versus autonomous control may be utilized in the system 1000, and embodiments disclosed herein may encompass fully user-controlled systems, fully autonomously-controlled systems, and systems having any combination of user and autonomous control. For operations that are user-controlled, the control system 1006 generates control signals in response to receiving a corresponding user input command via the user input and feedback system 1004. For operations that are autonomously controlled, the control system 1006 may execute pre-programmed logic (e.g., a software program) and may determine and send control commands based on the programming (e.g., in response to a detected state or stimulus specified in the programming). In some systems, some operations may be user controlled and others autonomously controlled. Moreover, some operations may be partially user controlled and partially autonomously controlled—for example, a user input command may initiate performance of a sequence of events, and then the control system 1006 may perform various operations associated with that sequence without needing further user input.

While the control system 1006 is illustrated as a separate element in FIG. 11, those having ordinary skill in the art would understand that the control system 1006 may be a distributed system, or portions thereof may be provided in and/or distributed among one or more of the auxiliary system 1008, the user input and feedback system 1004, and the manipulator assembly 1001.

The auxiliary system 1008 may comprise various auxiliary devices that may be used in operation of the system 1000. For example, the auxiliary system 1008 may include power supply units, auxiliary function units (e.g., functions such as irrigation, evacuation, energy supply, illumination, sensors, imaging, etc.). As one example, in a system 1000 for use in a medical procedure context, the auxiliary system 1008 may comprise a display device for use by medical staff assisting a procedure, while the user operating the input devices may utilize a separate display device that is part of the user input and feedback system 1004. As another example, in a system 1000 for use in a medical context, the auxiliary system 1008 may comprise flux supply units that provide surgical flux (e.g., electrical power) to instruments 1010. An auxiliary system 1008 as used herein may thus encompass a variety of components and does not need to be provided as an integral unit.

The embodiments described herein (including the instrument 10 and system 1000 described above) may be well suited for use in medical applications. In particular, some embodiments are suitable for use in, for example, surgical, teleoperated surgical, diagnostic, therapeutic, and/or biopsy procedures. Such procedures could be performed, for example, on human patients, animal patients, human cadavers, animal cadavers, and portions or human or animal anatomy. Some embodiments may also be suitable for use in, for example, non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments may also be used for benchtop procedures on non-living material and forms that are not part of a human or animal anatomy. Moreover, some embodiments are also suitable for use in non-medical applications, such as industrial robotic uses, and sensing, inspecting, and/or manipulating non-tissue work pieces. In non-limiting embodiments, the techniques, methods, and devices described herein may be used in, or may be part of, a computer-assisted medical system employing robotic technology such as the da Vinci@X, Xi, and SP Surgical Systems commercialized by Intuitive Surgical, Inc., of Sunnyvale, California. Those skilled in the art will understand, however, that aspects disclosed herein may be embodied and implemented in various ways and systems, including manually operated instruments and computer-assisted, teleoperated systems, in both medical and non-medical applications. Reference to the daVinci® Surgical Systems are illustrative and not to be considered as limiting the scope of the disclosure herein.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes may be made without departing from the spirit and scope of this description and the claims. In some instances, well-known circuits, structures, and techniques have not been shown or described in detail in order not to obscure the embodiments. Like numbers in two or more figures represent the same or similar elements.

In the description above, reference is made to “paying out” or “drawing in” actuation elements. As noted above, paying out or drawing in an actuation element refers to increasing or decreasing the length of a segment of the actuation element that extends between a take-off point on the drive member to which the cable is coupled and a coupling point at which the actuation element is operably coupled to the structure whose motion the actuation element drives. The take-off point on a drive member refers to the point at which the actuation element ceases to contact the drive member (or begins to contact the drive member, depending on the point of view). The take-off point of the drive member may move as the drive member is operated—for example, as a capstan is rotated and a actuation element is wound onto or wound off from the capstan, the take-off point on the capstan may move axially along the capstan. As noted above, the coupling point is a point at which the actuation element is operably coupled to the structure that the actuation element is configured to drive, such as a point at which the actuation element is coupled to a movable component (if the actuation element is to drive motion of the movable component), the point at which the actuation element is coupled to a shaft (if the actuation element is to drive translation of the shaft), the point at which the cable is coupled to another actuation element (if the actuation element is to drive motion of the other actuation element), and so on. For example, in the embodiment of FIG. 5, the coupling points 565_2 to 565_5 are illustrated, which are fixed to the articulable structures 230. In other embodiments, the coupling points may be different. A coupling point can constitute an end point of a given segment of the actuation element, which may be, but does not necessarily have to be, an end point of the entire actuation element. For example, in the embodiment of FIG. 5, the coupling points 565_2 to 565_5 constitute end points of the entire actuation elements 500_2 to 500_5. In other embodiments, one or more coupling points are not the end point of their respective actuation element—for example, the coupling point may be a point where a portion of the actuation element meets a pulley attached to the component driven by the actuation element. Moreover, the operable coupling of the actuation element to the structure it drives at the coupling point does not necessarily entail the actuation element being fixedly attached to the structure. Instead, the actuation element may be movably coupled to the structure it drives, for example via a pulley attached to the structure as previously mentioned.

In some circumstances, multiple portions of an actuation element may be coupled to the same drive member or to separate drive members. For example, one end of the actuation element may be coupled to a drive member, extend from the drive member to the structure driven by the actuation element, and then loop back such that an opposite end of the actuation element is coupled to a drive member (the same or different drive member). In such cases, there are two segments of the actuation element that extend between the drive member(s) and the coupling point. In such circumstances, references to “paying out” or “drawing in” the actuation element should be understood as referring to paying in or drawing out one of these segments of the actuation element. The particular segment that is being referenced may be understood from the context if not explicitly identified.

Further, the terminology used herein to describe aspects of the invention, such as spatial and relational terms, is chosen to aid the reader in understanding example embodiments of the invention but is not intended to limit the invention. For example, spatially terms-such as “beneath”, “below”, “lower”, “above”, “upper”, “proximal”, “distal”, “up”, “down”, and the like—may be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items may be posed differently.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least any inconsequential variations from the value, property, or relationship, such as variations that are equivalents to the stated value, property, or relationship. The range of variation around the stated value, property, or relationship also includes at least those variations that are typical in the relevant art for the type of item in question due to manufacturing or other tolerances.

As used herein, “transverse” refers to a positional relationship of two items in which one item is oriented crosswise at an angle relative to the other item, such as being substantially or generally perpendicular to the other item. As used herein, “transverse” includes, but does not require, an exactly perpendicular relationship. For example, unless otherwise noted herein or implied by the context, “transverse” may include at least positional relationships in which one item is oriented at nonparallel angle to the other item, such as for example, an angle ranging from 45° to 135° relative to the other item.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

What is claimed is:

1. A medical instrument comprising:
an instrument shaft comprising a proximal portion and a distal portion;
a movable component coupled to the distal portion of the shaft;
a first actuation element coupled to the instrument shaft and a second actuation element coupled to the movable component; and
a transmission assembly movably coupled to the instrument shaft and comprising a plurality of drive members, the plurality of drive members comprising:
a first drive member comprising a rotatable drum, the first and second actuation elements being at least partially wound around the rotatable drum and configured to drive translation of the instrument shaft relative to the transmission assembly in response to rotation of the drum;
a second drive member comprising a first rotatable drive shaft, the second actuation element being coupled to the second drive member and actuatable by rotation of the first rotatable drive shaft; and
a third drive member comprising a second rotatable drive shaft and a bearing coupled to the second rotatable drive shaft, the second actuation element being routed over the bearing between the rotatable drum and the first rotatable drive shaft.

2. The medical instrument of claim 1, wherein the bearing is rotatable relative to and coaxial with the second rotatable drive shaft.

3. The medical instrument of claim 1, wherein:
the transmission assembly further comprises a biasing device coupled to the second drive member and configured to generate a biasing force urging the first rotatable drive shaft to rotate to draw in the second actuation element.

4. The medical instrument of claim 3, comprising:
a third actuation element coupled to the movable component and to the third drive member, the third actuation element actuatable by rotation of the second rotatable drive shaft;
wherein the biasing device is coupled to the third drive member and configured to generate another biasing force urging the second rotatable drive shaft to rotate to draw in the third actuation element.

5. The medical instrument of claim 4, wherein:
the biasing device comprises a first torsion spring and a second torsion spring;
the first torsion spring is wrapped around and coupled to the second drive member; and
the second torsion spring is wrapped around and coupled to the third drive member.

6. The medical instrument of claim 5, wherein:
the biasing device further comprises a linkage portion coupling the first torsion spring and the second torsion spring together.

7. The medical instrument of claim 1, wherein:
the rotatable drum is rotatable in a first direction to drive translation of the instrument shaft in a distal direction relative to the transmission assembly; and
the rotatable drum is rotatable in a second direction, opposite the first direction, to drive translation of the instrument shaft in a proximal direction relative to the transmission assembly.

8. The medical instrument of claim 7, wherein:
the first actuation element has a first end affixed to the rotatable drum and a second end affixed to the proximal portion of the instrument shaft, and
each of the first and second actuation elements is coupled to the rotatable drum such that:
rotation of the rotatable drum in the first direction drives translation of the instrument shaft in the distal direction by drawing in the first actuation element while paying out of the second actuation element; and
rotation of the rotatable drum in the second direction drives translation of the instrument shaft in the proximal direction by paying out the first actuation element while drawing in the second actuation element.

9. The medical instrument of claim 1, wherein the transmission assembly comprises:
a housing comprising a chassis that supports the plurality of drive members, wherein the transmission assembly is removably mountable to an interface of an instrument manipulator;
a sleeve coupled to and extending distally from the chassis and surrounding the instrument shaft; and
a seal positioned between the sleeve and the instrument shaft.

10. The medical instrument of claim 9, wherein:
the second actuation element is routed external to the instrument shaft along the proximal portion of the instrument shaft;
the second actuation element is routed internal to the instrument shaft along the distal portion of the instrument shaft; and
the seal engages the proximal portion of the instrument shaft.

11. The medical instrument of claim 10, wherein:
the instrument shaft comprises an opening extending from an exterior surface of the instrument shaft to an interior of the instrument shaft;
the second actuation element passes between the proximal and distal portions of the instrument shaft through the opening; and
the opening is located between the seal and a distal end portion of the sleeve throughout a full range of motion of the instrument shaft relative to the transmission assembly.

12. The medical instrument of claim 1, wherein the transmission assembly further comprises a plurality of waterfall pulleys, each of the first and second actuation elements being respectively routed via one of the plurality of waterfall pulleys so as to extend proximally or distally along the instrument shaft.

13. The medical instrument of claim 1, wherein:
the second drive member comprises an actuation transfer mechanism coupled to the first rotatable drive shaft;
the second actuation element is routed over the actuation transfer mechanism;
the actuation transfer mechanism is configured such that the second actuation element is actuatable by rotation of the first rotatable drive shaft and the second actuation element is actuatable by the first drive member independently of rotation of the first drive shaft.

14. The medical instrument of claim 13, wherein the actuation transfer mechanism comprises:
a first pulley rotatable relative to the first rotatable drive shaft about a rotational axis parallel with a rotational axis of the first rotatable drive shaft;
a second pulley having a rotational axis transverse to the rotational axis of the first rotatable drive shaft, the second pulley coupled to the first rotatable drive shaft such that rotation of the first rotatable drive shaft causes revolution of the second pully around the first rotatable drive shaft; and
a third pulley having a rotational axis parallel to the rotational axis of the first rotatable drive shaft and rotatable relative to the second rotatable drive shaft.

15. The medical instrument of claim 13, further comprising:
a third actuation element coupled to the movable component;
wherein:
the actuation transfer mechanism is a first actuation transfer mechanism; and
the third drive member comprises a second actuation transfer mechanism coupled to the second rotatable drive shaft and to the third actuation element.

16. The medical instrument of claim 1, wherein the movable component comprises a component of an end effector or an articulable structure.

17. The medical instrument of claim 1, further comprising:
an end effector;
a fifth actuation element; and
a sixth actuation element
wherein the movable component comprises an articulable structure comprising a first joint and a second joint, the first and second joints configured to provide pitch and yaw degrees of freedom of motion, respectively, to the end effector, and
wherein the second and third actuation elements are coupled to the first joint and the fifth and sixth actuation elements are coupled to the second joint.

18. A method of manufacturing a medical instrument, comprising;
providing an instrument shaft comprising a proximal portion and a distal portion;
coupling a movable component to the distal portion of the shaft;
movably coupling a transmission assembly to the instrument shaft, the transmission assembly comprising a plurality of drive members, the plurality of drive members comprising:
a first drive member comprising a rotatable drum;
a second drive member comprising a first rotatable drive shaft; and
a third drive member comprising a second rotatable drive shaft and a bearing coupled to the second rotatable drive shaft;
coupling a first actuation element to the instrument shaft;
coupling a second actuation element to the movable component;
coupling the first and second actuation elements to the rotatable drum such that the first and second actuation elements are configured to drive translation of the instrument shaft relative to the transmission assembly in response to rotation of the drum;
coupling the second actuation element to the second drive member such that the second actuation element is actuatable by rotation of the first rotatable drive shaft; and
routing the second actuation element over the bearing between the rotatable drum and the first rotatable drive shaft.

19. The method of claim 18, further comprising:
coupling a first biasing element to the second drive member, the biasing element configured to generate a biasing force urging the first rotatable drive shaft to rotate to draw in the second actuation element.

20. The method of claim 19, further comprising:
coupling a third actuation element to the movable component and to the third drive member, the third actuation element actuatable by rotation of the second rotatable drive shaft; and
coupling a second biasing element to the third drive member, the second biasing element configured to generate another biasing force urging the second rotatable drive shaft to rotate to draw in the third actuation element.
